(19) United States
(12) Patent Application Publication
(10) Pub. No.: US 2024/0198337 A1
(43) Pub. Date: Jun. 20, 2024

(54) Bone Marrow on a Chip

(71) Applicant: NEW YORK UNIVERSITY, New York, NY (US)

(72) Inventors: Weiqiang Chen, New York, NY (US); Chao Ma, New York, NY (US)

(21) Appl. No.: 18/555,363

(22) PCT Filed: Apr. 15, 2022
(86) PCT No.: PCT/US2022/024949

§ 371 Date: Oct. 13, 2023

Related U.S. Application Data
(60) Provisional application No. 63/175,853, filed on Apr. 16, 2021.

(51) Int. Cl. B01L 3/00; G01N 33/50

(52) U.S. Cl. CPC B01L 3/502761; B01L 3/502746; G01N 33/5052; B01L 2200/16; B01L 2300/0816; B01L 2400/086

(57) ABSTRACT

The present invention provides devices that replicate bone marrow niche in a microfluidic chip. The devices can be used to model certain disease states related to bone marrow, such as leukemic bone marrow niche remission and relapse under various treatment conditions. The devices can be adapted to replicate bone marrow niche from patient-specific cells such that treatment conditions can be modeled and tailored to individual patients. In some embodiments, the devices are suitable for evaluating leukemia therapies on a patient-specific basis.

### CROSS REFERENCE TO RELATED APPLICATIONS

This application claims priority to U.S. Provisional Application No. 63/175,853, filed Apr. 16, 2021, which is hereby incorporated by reference herein in its entirety.

### STATEMENT REGARDING FEDERALLY SPONSORED RESEARCH OR DEVELOPMENT

This invention was made with government support under Grant No. CBET 1701322 from the National Science Foundation, Grant No. R35 GM133646 from the National Institute of General Medical Sciences, and Grant No. RO1 CA243001 from the National Cancer Institute. The government has certain rights in the invention.

### BACKGROUND OF THE INVENTION

B-cell acute lymphoblastic leukemia (B-ALL) is the most common cancer among children and characterized by the overproduction of immature and dysfunctional B-cell blasts within bone marrow (BM). Despite the significant progress achieved over the past decade with multi-drug chemotherapy regimens, allogeneic hematopoietic stem cell (HSC) transplantation and, most recently, CD19-targeted CAR (chimeric antigen receptor) T-cell immunotherapy, relapse is common after initial treatment and the leading cause of death for pediatric B-ALL patients (Pui C H et al., New England Journal of Medicine. 2004 Apr. 8; 350(15): 1535-48; Hunger S P et al., New England Journal of Medicine. 2015 Oct. 15; 373(16): 1541-52). Patients with refractory and relapsed B-ALL have a poor prognosis with a 5-year survival rate of about 10%, due largely to acquired resistance by the heterogeneity in the BM microenvironment and tumor genetics (Pui C H et al., New England Journal of Medicine. 2004 Apr. 8; 350(15): 1535-48; Hunger S P et al., New England Journal of Medicine. 2015 Oct. 15; 373(16): 1541-52; Terwilliger T et al., Blood cancer journal. 2017 June; 7(6): e577). Currently, there is limited prognostic information between such heterogeneity and therapeutic responses, such as ETV6-RUNX1⁺ B-ALL patients often have favorable outcomes, while Philadelphia chromosome-positive (Ph+) B-ALL patients are expected to have an unfavorable prognosis (Churchman M L et al., Cancer cell. 2015 Sep. 14; 28(3): 343-56). A clearer understanding of the microenvironmental evolution during leukemia pathogenesis and the heterogeneity of acquired chemo-resistance mechanisms from distinct B-ALL subtypes may provide novel therapeutic targets for optimized therapy for refractory and relapsed B-ALL patients.

The BM microenvironment is characterized by a complex milieu of evolving interactions among hematopoietic and non-hematopoietic niche cells to facilitate normal hematopoiesis. Upon leukemic initiation, B-ALL blasts transform their BM niches, dysregulating BM niche cell signaling to promote B-ALL pathogenesis and evade targeted therapies. Clinical ALL cases have reported abnormal vascular architecture in leukemic BM described by increased sinusoidal endothelial cell (EC) and perivascular mesenchymal stromal cells (MSCs) proliferation, microvascular density and vascular permeability, as well as altered endosteal (osteoblastic) niche that together result in abnormal HSC and progenitor development and the accumulation of leukemia-associated factors (Schmidt T et al., Hematology 2010, the American Society of Hematology Education Program Book. 2011 Dec. 10; 2011(1): 1-8). Studies have demonstrated that leukemia colonization is driven by CXCL12 (C-X-C Motif Chemokine Ligand 12)/CXCR4 (C-X-C Motif Chemokine Receptor 4) signals derived from the vascular niche (Passaro D et al., Cancer cell. 2015 Jun. 8; 27(6): 769-79; Pitt L A et al., Cancer cell. 2015 Jun. 8; 27(6): 755-68). Leukemic blasts have also been shown to engage perivascular stromal cells or endosteal osteoblasts via intercellular adhesion mediated by, such as very late antigen-4 (VLA-4), vascular cell adhesion molecule 1 (VCAM-1) and osteopontin (OPN) (Boyerinas B et al., Blood, The Journal of the American Society of Hematology. 2013 Jun. 13; 121(24): 4821-31; Jacamo R et al., Blood. 2014 Apr. 24; 123(17): 2691-702; Hsieh Y T et al., Blood, The Journal of the American Society of Hematology. 2013 Mar. 7; 121(10): 1814-8). In addition, other hematopoietic cells, such as monocyte, may also be involved in regulating the chemotherapeutic response of B-ALL, as well as other types of leukemia (Witkowski M T et al., Cancer Cell. 2020 Jun. 8; 37(6): 867-82; Lee Y et al., Blood, The Journal of the American Society of Hematology. 2012 Jan. 5; 119(1): 227-37; Giannoni P et al., haematologica. 2014 June; 99(6): 1078). Moreover, HSCs residing in either endosteal region or medullary cavity demonstrate distinct niche-regulated cell fates (e.g. proliferation, quiescence, and differentiation) (Ding L et al., Nature. 2013 March; 495(7440): 231-5), suggesting the perivascular and endosteal niches may differentially regulate B-ALL progression. These different niche cell components, cytokine and adhesive signaling together can promote leukemia survival and/or dormancy, yet the downstream regulators remain not fully defined with transcription nuclear factor-kappa B (NF-κB) being implied as a functional role (Boyerinas B et al., Blood, The Journal of the American Society of Hematology. 2013 Jun. 13; 121(24): 4821-31; Jacamo R et al., Blood. 2014 Apr. 24; 123(17): 2691-702; Hsieh Y T et al., Blood, The Journal of the American Society of Hematology. 2013 Mar. 7; 121(10): 1814-8). In addition, while the functions of perivascular and endosteal niches have been concurrent in healthy hematopoiesis and other leukemia types such as acute myeloid leukemia (AML), it remains poorly understood how B-ALL blasts may distinctly exploit BM microenvironment signaling to raise chemo-resistance within these different BM niches (Duan C W et al., Cancer cell. 2014 Jun. 16; 25(6): 778-93; Ebinger S et al., Cancer cell. 2016 Dec. 12; 30(6): 849-62; Duarte D et al., Cell stem cell. 2018 Jan. 4; 22(1): 64-77).

At present, pre-clinical ALL murine models, with the aid of intravital microscopy, have provided the foundation of the in vivo leukemic BM ecology. Results emanating from these approaches may be difficult to reproduce as demonstrated, for instance, in previous T-ALL studies highlighting differences in leukemic blast localization in vivo (Passaro D et al., Cancer cell. 2015 Jun. 8; 27(6): 769-79; Pitt L A et al., Cancer cell. 2015 Jun. 8; 27(6): 755-68; Hawkins E D et al., Nature. 2016 October; 538(7626): 518-22) and translate to molecularly-distinct B-ALL subtypes. The conventional two-dimensional (2D) or three-dimensional (3D) cell co-culture systems are simple and convenient platform for biological studies but they cannot recapitulate the key architectures and characteristics of the in vivo B-ALL BM niche such as the central sinus, medullar cavity, and endosteal space as well as the hematopoietic environment (Ma C et al., Trends in Pharmacological Sciences. 2020 Dec. 16; Bhatia S N et al., Nature biotechnology. 2014 August; 32(8): 760-72; Duarte D et al., Blood. 2018 Apr. 5; 131(14): 1507-11). Moreover, scanning cell compositions in leukemic BM niches provides an overall demographic of BM cell populations, but it does not map the real-time and dynamic evolution of the tumor-niche crosstalk. Recent advances in microfluidics-based ‘Organ-on-a-Chip’ in vitro models have been applied to mimic the pathophysiology of solid tumor microenvironments, yet few attempts have been made to accurately recapitulate the in vivo anatomical architectures and heterogeneity of the liquid tumors such as B-ALL, especially for revealing the heterogeneous resistance mechanisms (Zheng Y et al., Advanced healthcare materials. 2016 May; 5(9): 1014-24; Mannino R G et al., Lab on a Chip. 2017; 17(3): 407-14; Bruce A et al., PLoS One. 2015 Oct. 21; 10 (10): e0140506; Zhang W et al., Tissue Engineering Part C: Methods. 2014 Aug. 1; 20(8): 663-70).

There remains a critical unmet need for a reliable methodology to model the CAR T-cell therapy in patient-specific leukemic bone marrow immunity so as to reveal the mechanisms of relapse and predict patient responses prior to the administration of therapy. To dissect the heterogeneity of BM niche mechanisms associated with treatment resistance for genetically-distinct B-ALL subtypes, there is a critical demand for both an accurate, real-time, and modular methodology and reliable clinical biomarkers to identify and screen promising therapy targets for patients with refractory and relapsed B-ALL diseases. The present invention meets this unmet need.

### SUMMARY OF THE INVENTION

In one aspect, the present invention relates to a bone marrow on a chip device, comprising: a cartridge housing; a central chamber embedded in the cartridge housing; at least one aperture fluidly connected to the central chamber; and a plurality of evenly spaced micropillars arranged in a substantially circular shape within the central chamber such that the central chamber is partitioned into at least a first inner region and a first outer region; wherein the first inner region comprises endothelial cells configured to mimic a venous sinus and the first outer region comprises endothelial cells and mesenchymal stromal cells configured to mimic a medullary cavity.

In one embodiment, the first inner region and the first outer region are concentric. In one embodiment, the central chamber comprises an additional second outer region adjacent and concentric to the first outer region, the second outer region being defined by a plurality of evenly spaced micropillars arranged in a substantially circular shape and comprising osteoblasts configured to mimic an endosteal region. In one embodiment, the plurality of micropillars have a cross-sectional shape selected from the group consisting of: circular, ovoid, square, rectangular, triangular, trapezoidal, and polygonal. In one embodiment, the plurality of micropillars are evenly spaced by a distance between about 50 μm and 200 μm.

In one embodiment, the device further comprises one or more sensors comprising capture molecules or probes positioned within the central chamber. In one embodiment, the capture molecule or probe is selected from the group consisting of: antibodies, antibody fragments, antigens, proteins, nucleic acids, oligonucleotides, peptides, lipids, lectins, inhibitors, activators, ligands, hormones, cytokines, sugars, amino acids, fatty acids, phenols, and alkaloids. In one embodiment, the one or more sensors are positioned between each of the micropillars. In one embodiment, the one or more sensors are localized surface plasmon resonance nanoplasmonic biosensors.

In one embodiment, the device is configured to replicate or mimic a bone marrow disease or disorder state selected from the group consisting of: leukemia, myeloma, anemia, infection, poisoning, and physical injury. In one embodiment, a device replicating or mimicking a leukemia disease state comprises B-cell acute lymphoblastic leukemia (B-ALL) cells in the first outer region.

In one aspect, the present invention relates to a method of determining leukemia treatment responsiveness, comprising the steps of: providing a device of the present invention; administering a leukemia treatment to the central chamber; and determining leukemia treatment responsiveness based on a measured change in the central chamber.

In one embodiment, the leukemia treatment is a chemotherapeutic selected from the group consisting of: nilotinib, prednisone, vincristine, daunorubicin, doxorubicin, cytarabine, L-asparaginase, 6-mercaptopurine, methotrexate, cyclophosphamide, dexamethasone, and nelarabine. In one embodiment, the measured change is a quantity of live and dead B-ALL cells after 1-3 days treatment or more.

In one embodiment, the leukemia treatment is chimeric antigen receptor (CAR) T-cell therapy, and wherein the central chamber further comprises CAR T-cells in the first inner region. In one embodiment, each of the cells in the central chamber are autologous cells.

In one embodiment, the measured change is a percent of leukemia cells relative to total cell population in the central chamber that is 5% or less, indicating responsiveness to CAR T-cell therapy. In one embodiment, the measured change is a percent of leukemia cells relative to total cell population in the central chamber that is 25% or more, indicating non-responsiveness to CAR T-cell therapy. In one embodiment, the measured change is a decrease in CD19 expression in B-ALL cells, indicating non-responsiveness to CAR T-cell therapy. In one embodiment, the measured change is an increase in suppressor immune cells, indicating non-responsiveness to CAR T-cell therapy. In one embodiment, the measured change is a greater level of expression of ETV6-RUNX1 gene versus BCR-ABL gene in B-ALL cells, indicating responsiveness to CAR T-cell therapy. In one embodiment, the measured change is an increase in cytokine levels selected from the group consisting of: IFN-γ, TNF-α, IL-2, and GZMB; indicating responsiveness to CAR T-cell therapy. In one embodiment, the measured change is an increase in cytokine levels selected from the group consisting of: TGF-β, IL-10, M-CSF, and CCL2; indicating non-responsiveness to CAR T-cell therapy. In one embodiment, the measured change is an increase in surface markers selected from the group consisting of: CD154, CD69, and CD107a; indicating responsiveness to CAR T-cell therapy.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a schematic of an exemplary leukemia-on-a-chip device.

FIG. 2 depicts a schematic of an exemplary leukemia-on-a-chip device seeded with cells.

FIG. 3A through FIG. 3G depict the design and fabrication of the biomimetic leukemic BM niche platform. (FIG. 3A) A cartoon illustrating the device design. The unit is m. (FIG. 3B) A schematic demonstrating the fabrication process of the devices using photolithography and soft lithography. The device mold was fabricated by patterning photoresist onto a silicon wafer (1, 2, and 3) using a high-resolution mask printed from (FIG. 3A). Uncured PDMS (light blue) was cast onto a silicon master mold (black) and cured at 80° C. for 1 hr to produce a thick PDMS layer, punched with holes and bonded to cover glass for framing 3D hydrogel (4 and 5). (FIG. 3C) The cell loading process. First, the sacrificial gelatin hydrogel solution (12 mg/ml in DPBS) was injected into the central area and solidified at 4° C. for 15 min. Then, a mixture of ECs, MSCs, and B-ALL cells in fibrin solution (3 mg/ml in DPBS) containing 4 U/ml thrombin was infused into in the ring area and gelled at room temperature for 10 min. To comparatively study the perivascular and endosteal niche, a mixture of osteoblast and B-ALL cells in fibrin solution (3 mg/ml in DPBS) containing 4 U/ml thrombin was then loaded into the outer area by a gentle vacuum suction. Following the gelation, cell culture media was added into the four media reservoirs and the device was incubated at 37° C. for 10 min. The gelled gelatin will then become liquefied and be removed thereafter. At last, ECs will be seeded at the central area. The established devices were cultured and monitored during the 3-day experiment protocol. (FIG. 3D) The whole scanning of the murine leukemic BM niche. Niche cells (red), and leukemia cells (cyan). (FIG. 3E) The 3D view of the co-localization of murine leukemia blasts and niche cells. ECs (red), MSCs (cyan), and B-ALL cells (green). White arrow denotes the cell co-localization. (FIG. 3F) The 3D tube-like vessel formed by HUVECs in the device. HUVECs were stained green with FITC-CD31 (BioLegend, 303104). White arrow denotes the vessel channel. (FIG. 3G) The representative image showed that CXCR4+ murine B-ALL cells were colocalized with CXCL12+ niche cells.

FIG. 4A through FIG. 4G depicts modeling the leukemic bone marrow (BM) niche in a Leukemia-on-a-Chip platform. (FIG. 4A) The schematic demonstrating the design of Leukemia-on-a-Chip that consists of three functional regions. (FIG. 4B) The whole scan of the resultant leukemic BM niche system, where the enlarged area shows the co-localization of murine B-ALL cells within the perivascular niche. The bottom insert shows co-localization in the endosteal niche. (FIG. 4C) The Hematoxylin and Eosin (H&E) staining image of the in vivo murine leukemic BM niche, with the enlarged area from showing the co-localization of B-ALL cells respectively within the perivascular and endosteal niches. (FIG. 4D) The chemo-resistance was compared between the engineered human BM niches of ETV6-RUNX1+ REH and Ph+ SUP-B15 B-ALL. Each drug concentration had >3 experimental replicates. (FIG. 4E) The cytokine profiles from two B-ALL blasts with and without niche cells were quantified using membrane-based ELISA analysis. (FIG. 4F) The quantification of nuclear (nuc)/cytoplasmic (cyto) ratio of NF-κB in REH and SUP B-ALL within their respective niche models. The ratios for REH and SUP were manually measured from 3 experimental replicates (N>150). (FIG. 4G) The percentage of Ki67+ B-ALL cells, corresponding to (FIG. 4F). Data was collected from 3 experimental replicates. Unpaired t-test (**p<0.01, Mann-Whitney Test).

FIG. 5A through FIG. 5F depict scRNA-seq mapping of engineered human leukemic BM niches. (FIG. 5A) Study overview. (FIG. 5B) UMAP visualization of color-coded clustering of the leukemic niches. All cells can be grouped into five clusters: REH, SUP, hMSCs (MSC), HUVECs (EC) and osteoblasts (Osteo). (FIG. 5C) Genes signature of 5 clusters based on relative expression of the 20 most-significant markers. Key genes highlighted on bottom. (FIG. 5D) Expression levels of lineage-specific genes via UMAP representation. Dashed lines encompass the examined CD19+PAX5+ (black), CDH5+ (red), NES+ (cyan) and COL1A1+ (yellow) populations. The CD19+PAX5+ populations can be divided into two subpopulations based on PTPRC expression. (FIG. 5E) and (FIG. 5F) MSigDB Hallmark gene set enrichment analysis. (FIG. 5E) The significantly-enriched gene expression profiles that are related to TNFA signaling via NF-κB and inflammation response were present in both REH and SUP, while SUP but not REH significantly decreased expression of mitotic spindle and G2M checkpoint related gene sets in leukemia niche models. (FIG. 5F) Comparative analysis of EC, MSC, and Osteo. Niche cells from both leukemia niches augmented expression of epithelial mesenchymal transition, inflammatory response and TNFA signaling via NF-κB related gene sets. Dot size represents adjusted P-value (padj), with normalized enrichment score (NES) denoted by shade of color highlighted in legend FIG. 6A through FIG. 6D depict comparative analysis of subtype-specific leukemia and niche signaling. (FIG. 6A) Gene set enrichment analysis of the five clusters with gene signatures of murine BM niche cells to confirm the cell type identity of the input cells. V1 and V2 are vascular ECs; P1, P2, P3, and P4 are perivascular MSCs; and O1, O2, and O3 are endosteal osteoblasts. (FIG. 6B) Heatmap showing fold change of gene expression in REH and SUP B-ALL cells after culture with or without niche cells. (FIG. 6C) Gene set enrichment analysis (GSEA) on NF-κB signaling related genes. GSEA on NF-κB signaling related genes of REH B-ALL cells cultured with or without niche cells (Up). GSEA on NF-κB signaling related genes of SUP B-ALL cells cultured with or without niche cells (Bottom). (FIG. 6D) Heatmap showing fold change of gene expression in niche cells (hMSC, osteoblast, and HUVEC) from the REH and SUP BM niches.

FIG. 7A through FIG. 7E depict the results of real-time monitoring of the leukemia-niche cell interaction dynamics. (FIG. 7A) The dynamic migration of niche cells (ECs) towards leukemia cells during 3-day culture. The result was extracted from one representative images within 3 experimental replicates. Scale bar, 1 cm. (FIG. 7B) The evolving dynamics of the murine leukemic BM niche during 3-day culture. The intercellular distance between leukemia and niche cells were manually quantified (N>50) from 3 experimental replicates. The bin size at 5 was set for the histogram. (FIG. 7C) The clustering behavior of B-ALL cells cultured with or without niche cells. The B-ALL clusters (defined with at least 20 cells) were manually quantified from 3 experimental replicates. Unpaired t-test (**p<0.01, Mann-Whitney Test). (FIG. 7D) Migration trajectory of individual B-ALL cells (N=60) was recorded every 5 min over a period of 4 hours by time-lapse microscopy. In the flower plot diagram, the starting point of each track is placed at the axis origin. Data are representative of 3 individual experiments. (FIG. 7E) Average migration velocity (m/min) of B-ALL cells of the indicated conditions analyzed in (FIG. 7D). Unpaired t-test (n.s., not significant, **p<0.01).

FIG. 8A through FIG. 8J depicts the results of niche cells promoting leukemia progression via cytokine and adhesive signaling. (FIG. 8A) The regional CXCR4 distribution on murine B-ALL cells cultured with or without niche cells. (FIG. 8B) The quantified result corresponding to (FIG. 8A). (FIG. 8C) Membrane-based ELISA analysis of CXCL12 expression level of niche cells (ECs and MSCs). (FIG. 8D) The quantified result corresponding to (FIG. 8C). (FIG. 8E) The representative image showing B-ALL cells co-localized with niche cells via VCAM-1/VLA-4 signaling. (FIG. 8F) The representative images showing nuclear translocation of NF-κB in B-ALL cells. (FIG. 8G) NF-κB activation in B-ALL cells with or without niche cells and under treatments with CXCR4 inhibitor, AMD3100 (AMD) and VLA-4 inhibitor, BIO5192 (BIO). The ratios were manually quantified from 3 experimental replicates (N>200). One-way ANOVA followed by Tukey's post hoc test. (FIG. 8H) B-ALL cell viability cultured with or without niche cells. (FIG. 8I) The quantification of NF-κB activation in human NALM-6, 697, RS(4; 11), and UOCB1 blasts within their leukemia niches. The ratios were manually measured from 3 experimental replicates (N>150). (FIG. 8J) The quantification of NF-κB activation in patient-derived B-ALL blasts within engineered leukemia niches. The ratios for various B-ALL blasts were manually measured from 3 technical replicates (N>20). Unpaired t-test (n.s., not significant, *p<0.05, **p<0.01, Mann-Whitney Test).

FIG. 9A through FIG. 9G depict the results of mouse cytokine antibody array-based analysis of B-ALL cells and niche cells. (FIG. 9A) Cytokine profile of niche cells. (FIG. 9B) Cytokine profile of niche cells with B-ALL. (FIG. 9C) Cytokine profile of MSCs (FIG. 9D) Cytokine profile of MSCs with B-ALL. (FIG. 9E) Cytokine profile of ECs. (FIG. 9F) Cytokine profile of ECs with B-ALL. (FIG. 9G) Cytokine profile of B-ALL.

FIG. 10A and FIG. 10B depict the quantification results of cytokine secretion of B-ALL cells and niche cells, corresponding to FIG. 10A through FIG. 10G.

FIG. 11A and FIG. 11B demonstrate B-ALL cells adheres onto niche cells via VLA-4 signaling. (FIG. 11A) Experimental setup for testing murine B-ALL cell adhesion onto niche cells [i.e. ECs (C166), MSCs (OP9), and osteoblasts (MC3T3)] with or without adhesion blockade. B-ALL cells were plated into the dishes with niche cell monolayer and cultured for 24 hours, followed by five times washing with warm PBS. The images were then taken with 20× objective. (FIG. 11B) Adhered B-ALL cells were manually counted and plotted as cell number per filed. Each condition has 3 replicates with >10 images being counted. One-way ANOVA followed by Tukey's post hoc test (n.s., not significant, *p<0.05, **p<0.01).

FIG. 12A and FIG. 12B depict NF-κB activation in murine B-ALL cells. (FIG. 12A) The representative images showing the nuclear translocation of NF-κB in on-chip cultured B-ALL cells, corresponding to FIG. 8F. (FIG. 12B) The representative images showing the nuclear translocation of NF-κB in 2D-cultured B-ALL cells. Unpaired t-test (*p<0.05, Mann-Whitney Test) (N>30).

FIG. 13A through FIG. 13L depict the results of niche cells functioning disparately to regulate leukemia progression. (FIG. 13A) Membrane-based ELISA analysis of CXCL12 secretion of ECs (Top left) and MSCs (Top right). Western blotting of CXCL12 expression of ECs (Bottom left) and MSCs (Bottom right). (FIG. 13B) VCAM-1 expression of ECs and the quantification result (N>200). (FIG. 13C) OPN expression of 2D cultured MSCs and the quantification result (N>200). (FIG. 13D) The correlation between distance of B-ALL to MSCs and dye retaining ability of B-ALL (N>120). B-ALL with lower DiD intensity was excluded as indicated by the dash line. (FIG. 13E) and (FIG. 13F) The flow cytometric images showing DiD dye retained in B-ALL, i.e. low (Lo), middle (Mid), and high (Hi) intensity. (FIG. 13G) The representative images showing p21 expression in B-ALL located in the perivascular and endosteal niches (N=54). (FIG. 13H) B-ALL viability in the two niches treated with different drugs. (FIG. 13I) NF-κB activation in REH after co-cultured with hematopoietic cells. (FIG. 13J) REH viability cultured with hematopoietic cells after 48 hr treatment of 20 nM VCR. (FIG. 13K) The representative images of CD34+ HSPC within (REH) or without (Control) the leukemia niche at day 1 and 9. (FIG. 13L) The quantified number of CD34+ cells. Unpaired t-test (n.s., not significant, *p<0.05, **p<0.01, Mann-Whitney Test).

FIG. 14A through FIG. 14C depict adhesive signaling expression of murine niche cells 2D co-cultured with or without B-ALL cells. (FIG. 14A) VCAM-1 (vascular cell adhesion moleucle-1) expression of ECs. (FIG. 14B) OPN (Osteopontin) expression of ECs. (FIG. 14C) OPN expression of osteoblasts. Each condition had 3 experimental replicates and were manually quantified (N>200). Unpaired t-test (n.s., not significant, **p<0.01, Mann-Whitney Test).

FIG. 15A through FIG. 15H depict Ki67 staining of murine and human B-ALL cells. (FIG. 15A) and (FIG. 15B) Ki67 staining of 2D co-cultured murine B-ALL cells. (FIG. 15A) The representative images of Ki67+/− murine B-ALL cells co-cultured with ECs and MSCs, respectively. (FIG. 15B) The quantified results of Ki67+ B-ALL percentage. The Ki67 is in purple, nuclei in blue, and actin in green. Unpaired t-test (**p<0.01, Mann-Whitney Test). N>70. (FIG. 15C) and (FIG. 15D) Ki67 staining of on-chip cultured murine B-ALL cells. (FIG. 15C) The representative images of Ki67+/− murine B-ALL cells cultured respectively with or without niche cells in the leukemic niche chip. (FIG. 15D) The quantified results of Ki67+ B-ALL percentage. The Ki67 is in light blue and nuclei in red. Each condition had 3 experimental replicates being counted. Unpaired t-test (**p<0.01, Mann-Whitney Test). (FIG. 15E through FIG. 15H) Ki67 staining of human B-ALL cells cultured with or without niche cells in the leukemia chip. (FIG. 15E) The Ki67+ B-ALL percentage in 697 B-ALL. (FIG. 15F) The Ki67+B-ALL percentage in RS(4; 11) B-ALL. (FIG. 15G) The Ki67+ B-ALL percentage in UOCB1 B-ALL. (FIG. 15H) The Ki67+ B-ALL percentage in NALM-6 BALL. The Ki67 is in green, CD19 in magenta, CD31 in red, and nuclei in blue. Each condition had >3 experimental replicates. Unpaired t-test (n.s., not significant, *p<0.05, **p<0.01, MannWhitney Test).

FIG. 16A through FIG. 16C depict the viability of B-ALL cells located at different regions without coculture with niche cells, in response to 2-day treatment of different drugs, corresponding to FIG. 13H. (FIG. 16A) NIL, nilotinib, 1 μM; (FIG. 16B) PRE, prednisone, 1 μM; (FIG. 16C) VCR, vincristine, 0.1 μM. Unpaired t-test (n.s., not significant, Mann-Whitney Test).

FIG. 17A through FIG. 17D depict on-chip testing of co-targeting niche signaling to eradicate leukemic burden. (FIG. 17A) The schematic of the protective leukemia BM niche regulating leukemia progression and quiescence via CXCL12/CXCR4 cytokine signaling and VCAM-1/VLA-4/OPN adhesive signaling. (FIG. 17B) The chemo-resistance phenomenon was confirmed in the condition that B-ALL cells cultured in the leukemia BM niche treated with three typical drugs (i.e. nilotinib, NIL, 1 μM; prednisone, PRE, 1 μM; vincristine, VCR, 0.1 μM) (N=6). (FIG. 17C) The representative graphs showing quantified result corresponding to the VCR-treated groups in (FIG. 17D). The mean fluorescence intensity of GFP and DAPI of each B-ALL cell identified in Image J (NIH) was plotted after incubation at the respective conditions. (FIG. 17D) The graphs showed the percentage of B-ALL killing quantified for blockade of different niche singling. The respective control groups were AMD alone, BIO alone, and BAY alone treated groups. Data are mean±s.d.; collected from 3 individual experiments. One-way ANOVA followed by Tukey's post hoc test (n.s., not significant, *p<0.05, **p<0.01).

FIG. 18A through FIG. 18C depict the results of monitoring the human CAR T-cell dynamics in the leukemic BM model. Real-time monitoring of CAR T-cell (FIG. 18A) extravasation, (FIG. 18B) infiltration, (FIG. 18C) recognition and killing activity of CD19+ leukemia cells. Scale bars: 40 μm. To clearly visualize different cell types in the system, human B-ALL cell line was engineered to express GFP, while HUVECs was engineered to express RFP (pseudo color: blue). Before infusion into the system, CAR T-cells were labeled with DiD dye (pseudo color: red)

FIG. 19A through FIG. 19D depicts the results of pre-testing of human CAR T-cell functionality in the leukemic BM model. (FIG. 19A) CAR T-cell specifically killed CD19+ but not CD19− B-ALL cells. (FIG. 19B) CAR T-cell expanded after being activated by CD19+ leukemia. (FIG. 19C) Time-lapse monitoring of tumor burden during CAR T therapy. (FIG. 19D) CAR T-cell increased various cytokines, such as INF-γ and TNF-α, in response to activation.

FIG. 20A through FIG. 20D depicts the results of in situ and multiplexed nanoplasmonic cytokine detection in an “Obesity-on-a-Chip”. (FIG. 20A) LSPR sensing mechanism using gold nanorods. (FIG. 20B) Calibration curves of 4 cytokines with LSPR imaging technique and correlated with ELISA (FIG. 20C) Obesity-on-a-Chip with a central adipose tissue chamber surrounded by a LSPR cytokine sensing barcode array. Scale bar, 200 μm. (FIG. 20D) Barcode-based analysis of spatiotemporal cytokine secretions at different stages and conditions of adipose tissue inflammation.

FIG. 21A and FIG. 21B depict microfluidics-based in vitro leukemic BM niche and relapse model with (FIG. 21A) integrated leukemia-immunity-stroma interactions, (FIG. 21B) on-chip LSPR-based nanoplasmonic sensing barcode array

### DETAILED DESCRIPTION

The present invention provides devices that replicate bone marrow niche in a microfluidic chip, and associated methods of use. The devices can be used to model certain disease states related to bone marrow, such as leukemic bone marrow niche remission and relapse under various treatment conditions. The devices can be adapted to replicate bone marrow niche from patient-specific cells such that treatment conditions can be modeled and tailored to individual patients. In some embodiments, the devices are suitable for evaluating leukemia therapies on a patient-specific basis.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Bone Marrow on a Chip

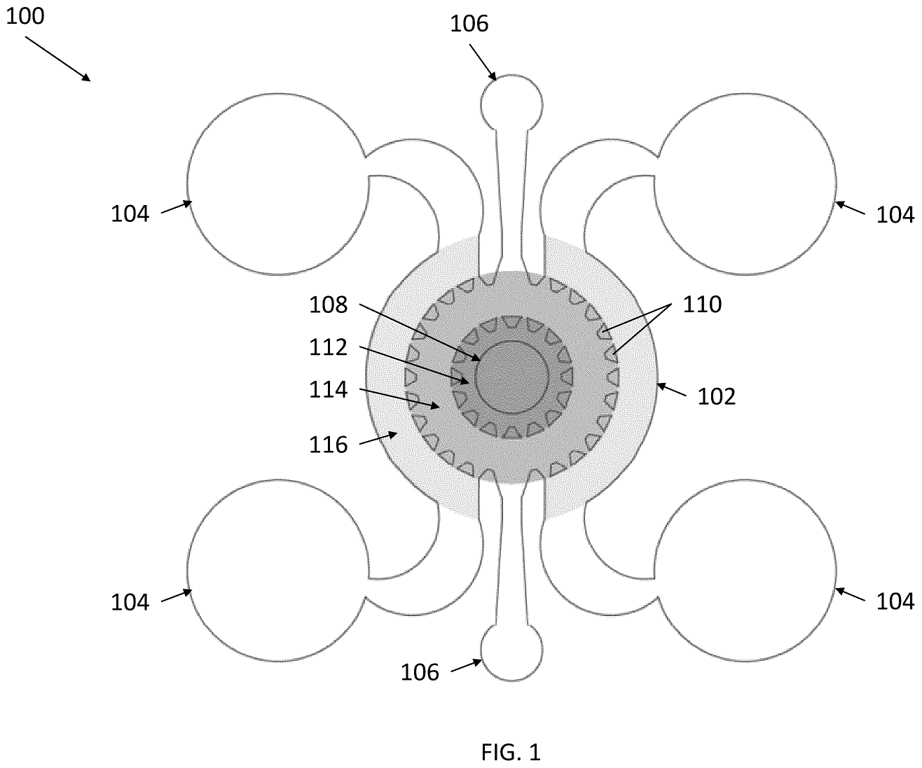

Referring now to FIG. 1, an exemplary layout of a bone marrow on a chip device 100 is depicted. Device 100 comprises a cartridge housing containing a central chamber 102 fluidly connected to one or more apertures. For example, central chamber 102 can be fluidly connected to one or more media apertures 104 and one or more cell apertures 106, wherein each fluid connection comprises one or more microchannels. In some embodiments, central chamber 102 can be directly connected to one or more apertures, such as aperture 108. While central chamber 102 is depicted in FIG. 1 as comprising four media apertures 104, two cell apertures 106, and a centrally positioned aperture 108, it should be understood that device 100 can comprise any desired number of apertures in any desired position or arrangement. The housing can be constructed from any desired material and can be at least partially transparent such that central chamber 102 is visible from an exterior of device 100.

Central chamber 102 comprises a substantially circular shape and receives one or more cells for co-culture. Central chamber 102 can be subdivided into a plurality of regions, wherein each region receives a population of cells to mimic native tissue architecture. Central chamber 102 can be partitioned into each of the regions by a series of micropillars 110. While micropillars 110 are depicted as having a trapezoidal cross-sectional shape, it should be understood that micropillars 110 can have any desired cross-sectional shape, including but not limited to circular, ovoid, square, rectangular, triangular, trapezoidal, polygonal, and the like. In the depicted embodiment, micropillars 110 are regularly-spaced by a distance configured to substantially impede flow of viscous materials such as hydrogel solutions, while permitting flow of liquid materials and diffusion of analytes through capillary action. Such a distance can be between about 50 μm and 200 μm.

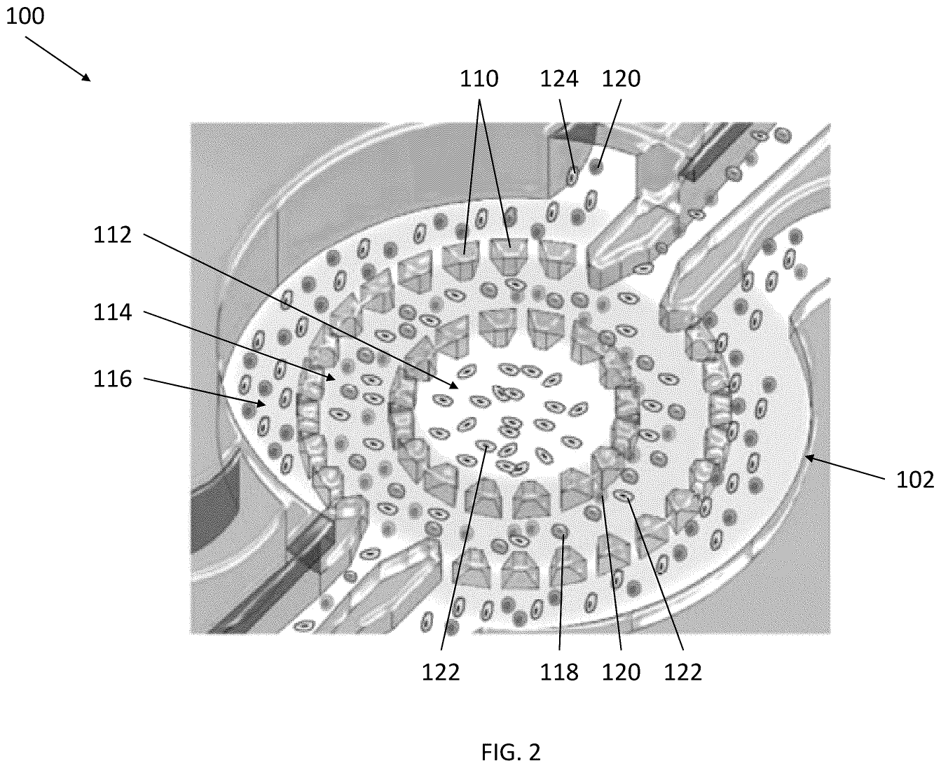

In some embodiments, central chamber 102 can comprise three concentric regions configured to mimic a bone marrow niche as depicted in FIG. 1: a central region 112 representing a venous sinus, a middle ring region 114 representing a medullary cavity, and an outer ring region 116 representing an endosteal region. Each of the regions can be correspondingly seeded with one or more populations of cells. For example, FIG. 2 depicts: central region 112 seeded with vascular endothelial cells 122; middle ring region 114 seeded with mesenchymal stromal cells 118 and vascular endothelial cells 122; and outer ring region 116 seeded with osteoblasts 124. In certain embodiments, apertures of device 100 are preferentially fluidly connected to a partitioned region. For example, FIG. 1 depicts media apertures 104 being preferentially fluidly connected to outer ring region 116, cell apertures 106 being preferentially fluidly connected to middle ring region 114, and aperture 108 being preferentially fluidly connected to central region 108.

In various embodiments, device 100 can be used to replicate or mimic bone marrow niche under certain disease or disorder states. Contemplated disease or disorder states include but are not limited to: leukemia, myeloma, anemia, infection, poisoning, physical injury, and the like. In such states, device 100 can be used to model the progression of a disease or disorder as well as evaluate therapies to treat a disease or disorder.

Cells may be isolated from a number of sources, including, for example, biopsies from living subjects and whole-organs recovered from cadavers. In some embodiments, the isolated cells are autologous cells obtained by biopsy from a subject, such as a cancer patient. Autologous cells can be used in device 100 to model progression and therapy on a patient-specific basis. In certain embodiments, the cells may be derived from cultured cell lines. In certain embodiments, the cells seeded into the device are differentiated from stem cells.

Seeding of cells into device 100 may be performed in any desired method. In one embodiment, the cells are embedded in a hydrogel solution and injected into a corresponding region of central chamber 102 by way of the one or more apertures. Injection of hydrogel solution may be accompanied by the application of a gentle vacuum at an oppositely positioned aperture to encourage infiltration of hydrogel solution into a respective region. Contemplated hydrogel solutions include but are not limited to fibrinogen, collagen, hyaluronic acid, alginate, polyacrylamide, polyethylene glycol, and the like. The hydrogel solution can be cross-linked within central chamber 102 based the material used, such as by photo-cross-linking, thermal-cross-linking, chemical cross-linking, and the like.

In some embodiments, device 100 further comprises one or more sensors for rapid analyte detection. The one or more sensors can comprise any desired sensing mechanism commonly used in art, including but not limited to chemically active regions, electrochemical sensors, immobilized capture molecules, probes, and the like. Contemplated probes or capture agents can be any suitable molecule, including antibodies, antibody fragments, antigens, proteins, nucleic acids, oligonucleotides, peptides, lipids, lectins, inhibitors, activators, ligands, hormones, cytokines, sugars, amino acids, fatty acids, phenols, alkaloids, and the like. The probes or capture agents can be configured to capture any desired molecule, including proteins, amines, peptides, antigens, antibodies, nucleic acids, steroids, eicosanoids, DNA sequences, RNA sequences, bacteria, viruses, and fragments thereof.

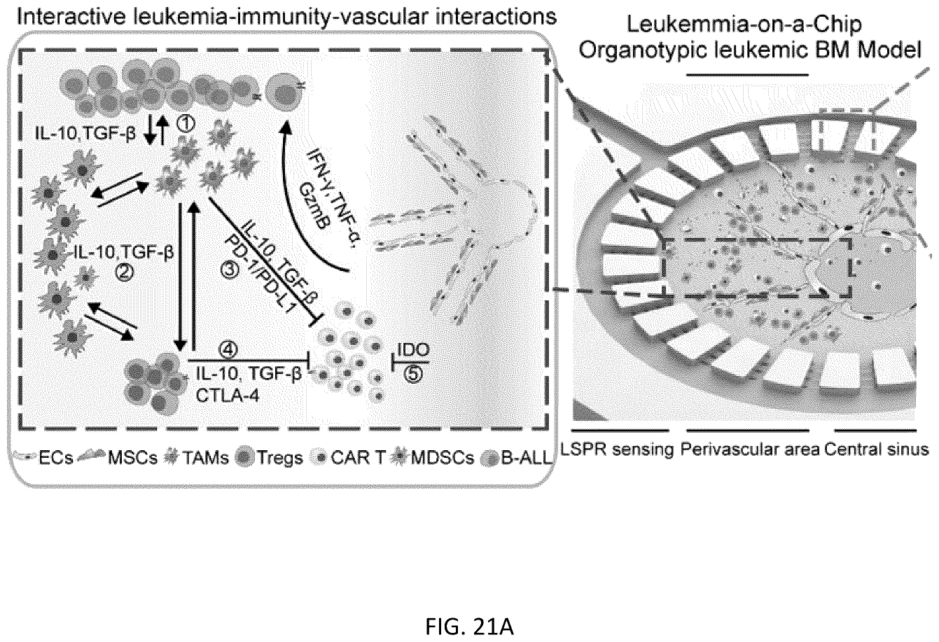

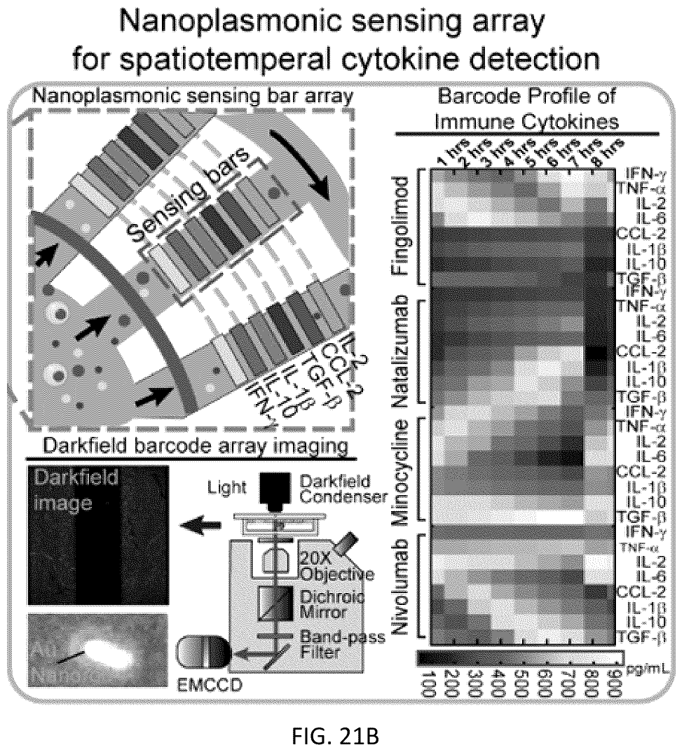

In some embodiments device 100 comprises label-free localized surface plasmon resonance (LSPR)-based nanoplasmonic biosensors, such as those depicted in FIG. 21A and FIG. 21B. The sensors can be arranged in array patterns and be configured to detect analytes relevant to disease or disorder progression and treatment in a replicated bone marrow niche. While the sensors are depicted in FIG. 21A and FIG. 21B to be positioned in spaces between micropillars, it should be understood that device 100 can have any desired number, size, shape, and arrangement of sensors.

The bone marrow on a chip devices of the present invention can be made using any suitable method known in the art. The method of making may vary depending on the materials used. For example, components substantially comprising a metal may be milled from a larger block of metal or may be cast from molten metal. Likewise, components substantially comprising a plastic or polymer may be milled from a larger block, cast, or injection molded. In some embodiments, the components may be made using 3D printing or other additive manufacturing techniques commonly used in the art. In some embodiments, microstructures and patterns can be achieved through microfabrication techniques including but not limited to: lithography, thin film deposition, electroplating, etching, micromachining, and the like.

### Leukemia-On-a-Chip

As described elsewhere herein, the bone marrow on a chip devices of the present invention can be used to model a variety of disease or disorder states in bone marrow, such as leukemia. Accordingly, the present invention further comprises methods of fabricating leukemia-on-a-chip devices and methods of characterizing leukemia treatment using the leukemia-on-a-chip devices. In some embodiments, leukemia bone marrow niche can be replicated or mimicked by providing leukemia cells, including but not limited to T-cell acute lymphoblastic leukemia cells, B-cell acute lymphoblastic leukemia cells (B-ALL), acute monocytic leukemia cells, acute myeloblastic leukemia cells, acute myelogenous leukemia cells, acute promyelocytic leukemia cells, basophilic leukemia cells, hairy cell leukemia, and the like. In some embodiments, a method of the present invention can include a step of providing leukemia cells from a source, wherein the source can be a tissue bank, an autologous source, an allogeneic source, or a xenogeneic source. In some embodiments, a method of the present invention can include a step of modifying the provided leukemia cells. In some embodiments, a method of the present invention can include a step of providing device 100 seeded with cells as described elsewhere herein to replicate or mimic a bone marrow niche, and further seeding middle ring region 114 and outer ring region 116 with one or more leukemia cells (such as B-ALL cells 120). The leukemia bone marrow niche can be used to evaluate the effectiveness of anticancer therapies, including but not limited to chemotherapy, radiation therapy, and immunotherapy. Accordingly, in some embodiments a method of the present invention can include a step of applying one or more leukemia treatments to a leukemia-on-chip device and a step of characterizing the effect of the one or more leukemia treatments on leukemia cells on the leukemia-on-a-chip device.

In some embodiments, device 100 adapted to replicate or mimic leukemia bone marrow niche can be used to evaluate chemotherapeutic responsiveness, including but not limited to nilotinib, prednisone, vincristine, daunorubicin, doxorubicin, cytarabine, L-asparaginase, 6-mercaptopurine, methotrexate, cyclophosphamide, dexamethasone, and nelarabine. Chemotherapeutic responsiveness can be evaluated over a period of 1-3 days or more. Chemotherapeutic responsiveness can be rated based on the number or percentage of live and dead B-ALL cells.

In some embodiments, device 100 adapted to replicate or mimic leukemia bone marrow niche can be used to evaluate chimeric antigen receptor (CAR) T-cell therapy, such that a method of the present invention includes a step of providing T-cells from a source (tissue bank, autologous, allogeneic, or xenogeneic as described elsewhere herein), a step of modifying the T-cells to express a chimeric antigen receptor and/or other receptors, and a step of seeding central region 112 with CAR T-cells and outer ring region 116 with bone marrow mononuclear cells. Progression of CAR T-cell therapy, in particular responsiveness to CAR T-cell therapy in the case of a personalized device 100 comprising autologous CAR T-cells, B-ALL cells, and bone marrow mononuclear cells, can be assessed by monitoring one or more of T-cell extravasation, migration, activation, expansion, and cytotoxicity.

CAR T-cells may be generated using any method known in the art. For example, in certain aspects T cells are isolated or obtained from a subject and genetically modified to express a CAR. In one embodiment, the T cells are modified by introducing a nucleic acid molecule (e.g., DNA, cDNA, or RNA) into the cell, wherein the nucleic acid molecule comprises a coding region encoding a CAR. In certain embodiments, either before or after genetic modification, the T cells may be expanded or activated, using methods known in the art.

CAR T-cells used in the devices and methods of the invention may express any type of CAR known in the art, or contemplated in the future. In certain embodiments, the CAR comprises an extracellular domain, a transmembrane domain, and a cytoplasmic domain. In one embodiment, the extracellular domain comprises an antigen binding domain. For example, in one embodiment, the antigen binding domain comprises a protein, peptide, antibody, or antibody fragment that binds to an antigen. In one embodiment, the antigen is a tumor-associated antigen or tumor-specific antigen. In one embodiment, the antigen binding domain comprises a protein, peptide, antibody, or antibody fragment that binds to an antigen associated with leukemia. In one embodiment, the cytoplasmic domain comprises one or more costimulatory or signaling domains.

A method of the present invention can include a step of characterizing CAR T-cell therapy in the leukemia-on-a-chip device. In some embodiments, CAR T-cell therapy is evaluated over a period of 1-4 weeks or more. In some embodiments, CAR T-cell therapy responsiveness can be rated based on observed leukemia remission or relapse, wherein remission can be described as a measure of leukemia cells relative to total cell population in device 100 of about 5% or less, and wherein relapse can be described as a measure of leukemia cells relative to total cell population in device 100 of about 25% or more. In some embodiments, CAR T-cell therapy responsiveness can be rated based on a percentage of CD19⁻ B-ALL cells, wherein an increase in CD19⁻ B-ALL cells (and therefore a decrease in CD19 expression) indicates trending towards relapse and non-responsiveness to CAR T-cell therapy. In some embodiments, CAR T-cell therapy responsiveness can be rated based on an accumulation of suppressor immune cells, wherein an increase in the presence of suppressor immune cells (e.g., CD16+ non-classical monocytes, regulatory T-cells (Treg), tumor associated macrophages (TAM), and myeloid derived suppressor cells (MDSC)) indicates trending towards non-responsiveness to CAR T-cell therapy. In some embodiments, CAR T-cell therapy responsiveness can be rated based on a viability test, wherein viability of B-ALL cells decreasing from an initial state of about 100% to about 5% or less indicates trending towards responsiveness to CAR T-cell therapy. In some embodiments, CAR T-cell therapy responsiveness can be rated based on cytokine production. For example, increased levels (about a 2 fold increase or more over an initial state) of cytokines including but not limited to IFN-γ, TNF-α, IL-2, and GZMB can indicate trending towards responsiveness to CAR T-cell therapy, while increased levels (about a 2 fold increase or more over an initial state) of cytokines including but not limited to TGF-β, IL-10, M-CSF, and CCL2 can indicate trending towards non-responsiveness to CAR T-cell therapy. For example, increased levels (about a 2 fold increase or more over an initial state) of surface markers including but not limited to CD154, CD69, and CD107a can indicate trending towards responsiveness to CAR T-cell therapy. Accordingly, treatment profiles that target cytokine production may be evaluated, such as the administration of neutralizing antibodies (e.g., 1D11, JES052A5, MAB416, and AB-479-NA).

### Bone Marrow on a Chip Kits

The present invention also provides kits for replicating or mimicking bone marrow niche. The kits include the bone marrow on a chip devices described elsewhere herein, as well as relevant reagents and instrumentation. For example, in some embodiments, the kit can comprise reagents for loading and culturing cell populations, including but not limited to hydrogels for 3D cell culture, cell culture media, wash media, and the like. In some embodiments, the kit can comprise instrumentation for manipulating contents of the bone marrow on a chip devices, including but not limited to pipettes, pipette tips, syringes, and the like. In some embodiments, the kit can comprise one or more capture molecules or probes as described elsewhere herein, wherein a user can select the one or more capture molecules or probes for inclusion in the sensors of the bone marrow on a chip devices to detect and/or quantify one or more analytes of interest.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out exemplary embodiments of the present invention, and are not to be construed as limiting in any way the remainder of the disclosure.

### Example 1: Leukemia-On-a-Chip—Dissecting the Chemo-Resistance Mechanisms in B-Cell Acute Lymphoblastic Leukemia (B-ALL) Bone Marrow Niche

The present study presents a novel 3D organotypic ‘Leukemia-on-a-Chip’ microphysiological system that maps the in vivo pathophysiology and heterogeneity of leukemic BM niches. Using this biomimetic system, the mechanistic details of the real-time, dynamic interactions between B-ALL blasts and its leukemic BM niche are accurately characterized within in vitro central sinus, medullary cavity and endosteum anatomical regions as well as the hematopoietic environment. The B-ALL subtype-specific niche signals are also comparatively mapped with the integration of single-cell RNA sequencing (scRNA-seq) to further dissect the heterogeneity of leukemic niches using various genetically-distinct human B-ALL cell lines and patient samples, and validated that the niche-enhanced downstream NF-κB signaling and cellular quiescence in B-ALL blasts promote chemotherapy resistance. Finally, the pre-clinical utility of the in vitro bioengineered model is demonstrated as a proof-of-concept to screen niche-co-targeting regimens, which together may translate to patient-specific therapeutics screening and disease management.

The materials and methods are now described.

### Cell Culture

Human umbilical vein endothelial cells (HUVEC, Lonza, C2519A) were cultured in EGM™-2 Endothelial Cell Growth Medium-2 (Lonza, CC-3162). Human bone marrow stem cells (hMSCs, Lonza, PT2501) were cultured in MSCGM™ Mesenchymal Stem Cell Growth Medium (Lonza, PT-3001). Human osteoblasts, hFOB 1.19 (hFOB, ATCC) were cultured using a 1:1 mixture of Ham's F12 Medium and Dulbecco's Modified Eagle's Medium (DMEM/F12) with 0.3 mg/ml G418 (Corning) and 10% heat inactivated fetal bovine serum (FBS, Invitrogen). All the primary cells were used in experiments between passages 3 and 8. Human cord blood CD34⁺ cells (Cat #70008.5) and human bone marrow mononuclear cells (Cat #70001.2) were purchased from STEMCELL Technologies and cultured in StemSpan™ SFEM II supplemented with StemSpan™ CC100. Human B-ALL cells [i.e. EVT6-RUNX1 REH (ATCC), MLL RS(4; 11) (ATCC), E2A-PBX1 697, E2A-HLF UOCB1 and NALM-6] were cultured in Roswell Park Memorial Institute (RPMI) 1640 medium (GIBCO) supplemented with 10% FBS, and Ph+ SUP-B15 cells (ATCC) in Iscove's Modified Dulbecco's Medium (IMDM, GIBCO) with 15% FBS. Patient-derived sample (Ph+ B-ALL blasts, PAUZUW) was sorted on the basis of CD45^{lo/mid}CD19⁺CD10⁺ (Witkowski M T et al., Cancer Cell. 2020 Jun. 8; 37(6): 867-82). Patient-derived samples (Ph+ B-ALL blasts, 16-265 and non-Ph+ B-ALL blasts, 16-656) were purchased from AMSBIO LLC, and isolated using EasySep™ Release Human CD19 Positive Selection Kit (STEMCELL). Since multiple cells are cultured in the microfluidic system, the culture media was used in a mixture (2:1:1:1) of HUVEC medium (EGM™-2, Lonza), hMSCs medium (MSCGM™, Lonza), hFOB medium (DMEM/F12) and Human B-ALL cell medium (RPMI1640 or IMDM, GIBCO).

Murine EC cell line, C166 (ATCC), derived from mouse yolk sac, was grown in Dulbecco's Modified Eagle Media (DMEM, Sigma), supplemented with 10% FBS and 1% penicillin/streptomycin. Murine MSC cell line, OP9 (ATCC), was grown in Minimum Essential Medium α (MEM-α, Thermo Fisher Scientific), supplemented with 20% FBS and 1% penicillin/streptomycin. Murine B-ALL cells was isolated from a well-characterized model of pediatric Ph+ B-ALL (Li S et al., The Journal of experimental medicine. 1999 May 3; 189(9): 1399-412), in which lethally-irradiated C57BL/6 mice are reconstituted with retrovirally-infected hematopoietic stem and progenitor cells ectopically co-expressing the B-ALL-associated P190 BCR-ABL1 isoform, as well as GFP for fluorescent cell tracing (Addgene Plasmid #38185). Following the isolation, Ph+ GFP+ B-ALL cells were cultured and expanded in IMDM supplemented with 15% FBS, 1% penicillin/streptomycin, 100 μM L-glutamine and 50 μM β-mercaptoethanol in a 37° C. (with 5% CO₂) incubator. In addition, to clearly distinguish the coexistence of ECs and MSCs, the two types of cells were pre-stained respectively with CellTracker Red CMTPX Dye (Thermo Fischer Scientific; 10 μM in DMEM, 45 min) and DiD dye (Thermo Fischer Scientific; 1:200 dilution in MEM-α, 20 min), prior to be loaded into the device for the subsequent studies.

### Hydrogel Solutions

The gelatin solution (12 mg/ml) was prepared by dissolving gelatin powder from porcine skin (G2500, Sigma) in 1× Dulbecco's PBS (DPBS without calcium and magnesium, Invitrogen), warming and vigorously stirring at 60° C. for 30 min. The gelatin solution was then sterile-filtered, aliquot and stored at 4° C. for future use. The fibrinogen solution (6 mg/mL) was prepared by dissolving lyophilized fibrinogen from bovine plasma (F8630, Sigma) in DPBS at 37° C. for 2 hr. The sterile-filtered fibrinogen solution was stored at 4° C. for future use within one week. The thrombin solution was prepared by reconstituting lyophilized thrombin (604980, Sigma-Aldrich) in sterile DPBS to 100 U/mL and stored in aliquots at −20° C.

### Device Design, Fabrication, and Cell Loading

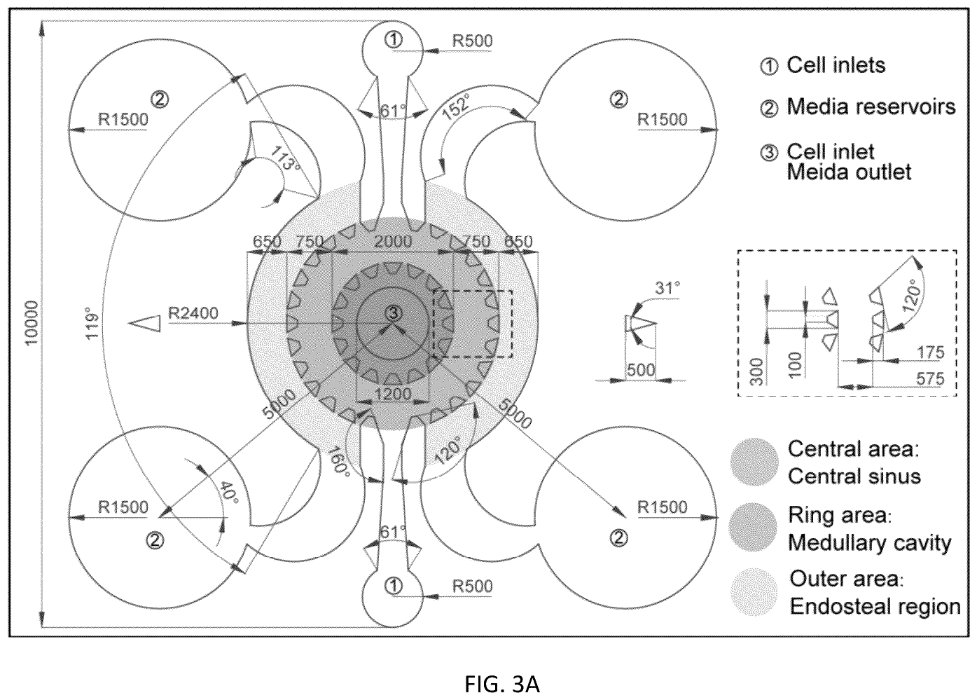

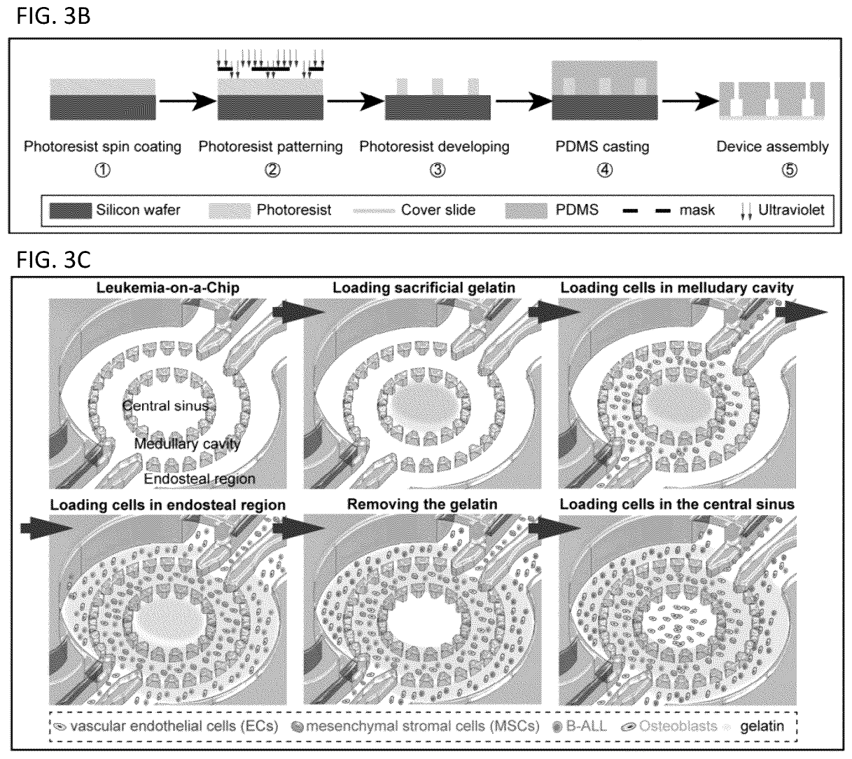

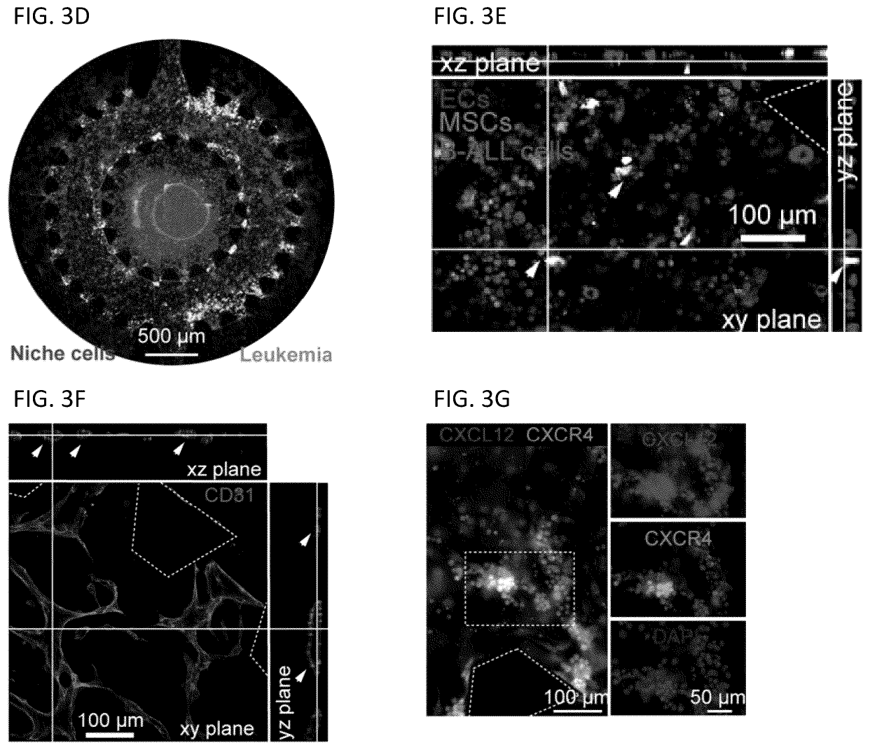

The 3D microfluidics-based organotypic ‘Leukemia-on-a-Chip’ device is composed of three distinct functional regions (FIG. 3A): a central sinus region vascularized by endothelial cells (ECs), an inner ring region serves as an interface of leukemia blasts (B-ALL cells) and niche cells (ECs and MSCs) interactions, and the outer ring channels (which is used to create the endosteal region by encapsulating osteoblasts and B-ALL cells within hydrogel), connected with four media reservoirs, for cell culture media supplies and waste removal. All the cell types were embedded in a fibrin hydrogel to maintain 3D cell culture. The microfluidic device was fabricated using standard soft lithography replica molding technique (Ma C et al., Analytical chemistry. 2016 Feb. 2; 88(3): 1719-27; Ma C et al., Lab on a Chip. 2016; 16(14): 2609-17). Generally, the mold for the microfluidic device was fabricated with SU-8 negative photoresist (2050, Microchem) at a thickness of 100 m on a silicon wafer by employing photolithography (FIG. 3B). Prior to the replica molding process, the SU-8 mold was surface-modified by trichloro (1H,1H,2H,2H-perfluorooctyl) (448931, Sigma-Aldrich) vapor overnight in vacuum desiccation to facilitate later PDMS (polydimethylsiloxane) release. Then, a mixture of PDMS base and curing agent (Sylgard 184, Dow Corning) at 10:1 weight-to-weight (w w) ratio were well mixed, cast on the mold, degassed, and then solidified in an 80° C. oven for 1 hr. Once PDMS was peeled from the mold, 1 mm and 3 mm holes were punched for three inlets and four media reservoirs, respectively. The cleaned PDMS slabs were finally bounded onto glass coverslips (22×22 mm; Thermo Fischer-Scientific) to assemble the microfluidic device using oxygen plasma (PE50XL, Plasma Etch; 350 W, 2 min) and then incubated overnight in an 80° C. oven for the recovery of hydrophobicity. The microfluidic devices were subsequently treated to ultraviolet for sterilization in a Type 2 class laminar flow hood for 20 min. Afterward, B-ALL cells, ECs and MSCs were embedded into 3D biomimetic hydrogels (fibrin, 3 mg/mL) (Carrion B et al., Tissue Engineering Part C: Methods. 2014 Mar. 1; 20(3): 252-63), following a multi-step loading protocol to seed ECs in the central sinus region and encapsulate B-ALL cells and niche cells (e.g. ECs and MSCs) in the medullary cavity region (FIG. 3C). The seeding densities of ECs, MSCs, and B-ALL cells in medullary cavity region were 2.5×10⁶ cells/ml, 2.5×10⁶ cells/ml, and 5×10⁶ cells/ml, respectively. To comparatively study the perivascular and endosteal niches, a mixture of osteoblasts and B-ALL cells (both at 5×10⁶ cells/ml) in 3 mg/ml fibrin solution was also loaded into the outer ring area to form the endosteal region and perivascular/endosteal niche interface. The cell-laden chips were cultured for three days, after which the morphology was analyzed by immunostaining. Note that the cell-laden chips for the human system were cultured for about seven days.

### Cell Motility

GFP+ B-ALL cells, C166 ECs and OP9 MSCs were seeded in the biomimetic device and allowed to balance for 4 hr or culture for 48 hr at 37° C. with 5% CO₂. After the defined incubation, the device was mounted on an inverted phase contrast microscope (Zeiss Axio Observer.Z1) with a motorized stage and an environment control incubation chamber (Incubator XLmulti S1) to maintain 37° C. with 5% CO₂. Phase contrast and fluorescent images were recorded every 5 min for 4 hr using a digital CMOS camera (ORCA-Flash4.0 LT, Hamamatsu Photonics) with a 20× objective. Each single cell was manually labeled in the continuous frames (in total, 49 frames) for 4 hr. Cell motility parameters were assessed via tracking of single B-ALL cell (60 cells per conditions) in ImageJ (NIH) using Manual Tracking plug-in. Average cellular migration speed was defined by the distance traveled in a unit time calculated using the corresponding x and y coordinates at initial time tₙ₋₁ and end time tₙ.

### Immunoassays

Cytokine secretion profiles of niche cells were examined by using Mouse Cytokine Antibody Array membrane-based ELISA kit (AAM-CYT-3, Ray Biotech) or Human Cytokine Antibody Array membrane-based ELISA kit (AAH-CYT-1, Ray Biotech), according to the manufacturer's protocols. Briefly, supernatants were collected after 48 hr culture and centrifuged at 2,000 r/min for 20 min at 4° C. to remove cellular debris, and then incubated overnight with Cytokine Antibody Array membranes. Biotinylated Antibody Cocktail was incubated with the membranes at 4° C. overnight, followed by washing and incubation with HRP-labeled Streptavidin (1:1000) at 4° C. overnight. Detection Buffer C and D mixture (1:1) was then applied to visualize chemiluminescence for 2 min at room temperature. Imaging was obtained by using a ChemiDoc Imaging System (Bio-rad). Mean intensity of each spot was quantified in ImageJ (NIH) using Protein Array Analyzer plug-in (written by Gilles Carpentier, Faculté des Sciences et Technologies, Université Paris, Paris, France).

### Western Blotting

Niche cells were cultured alone or co-cultured with B-ALL cells for 3 days. The cells were lysed in RIPA cell lysis buffer, supplemented with Halt Protease and Phosphatase Inhibitor Cocktail (1:100, Thermo Fisher-Scientific) for 30 min on ice, then centrifuged at 13,000 r/min for 20 min at 4° C., and the supernatant was stored at −80° C. until assayed. Protein content was determined with Protein Quantification Kit-Rapid (51254, Sigma-Aldrich). Proteins (30 μg) were loaded on Mini-PROTEAN® TGX Stain-Free™ Protein Gels (4568123, Bio-Rad) and then electroblotted onto polyvinylidene difluoride (PVDF) transfer membranes (1704156EDU, Bio-Rad). After blocking with 5% (w/v) non-fat dry milk in Tris buffered saline with 0.1% Tween 20 (TBST) for 1 hr at RT, the membranes were incubated overnight at 4° C. with CXCL12 antibody (1:1000, 3740S, Cell Signaling Tech), with GAPDH (1:1000, 2118S, Cell Signaling Tech) used as a housekeeping gene. After three-time washing with TBST for 30 min, the membranes were incubated for 1 hr at RT with a goat anti-rabbit IgG horseradish peroxidase (HRP)-conjugated polyclonal antibody (1:4000; Bio-Rad), and developed with a chemiluminescence enhancement kit (Clarity Max Western ECL Substrate, 1705061, Bio-Rad). Band densities were quantified from digital acquisition by a Chemidoc Imaging System (Bio-Rad) in ImageJ (NIH) using Gels plug-in.

### Histopathology

The in vivo Ph+ B-ALL murine model was generated using a previously published approach and GFP+ leukemic blasts were intravenously injected into non-irradiated C57BL/6 syngeneic recipient mice (Li S et al., The Journal of experimental medicine. 1999 May 3; 189(9): 1399-412). When leukemic burden reached approximately 20% in total BM cells (about 12 days post-transplantation), B-ALL recipient mice were sacrificed, and femurs were fixed overnight in 4% paraformaldehyde (PFA) at 4° C. For H&E sectioning, fixed femurs were decalcified in 14% EDTA for 48 hr prior to be dehydrated in 70% ethanol and embedded in paraffin. Paraffin sections in 5 μm were stained with Hematoxylin and Eosin (H&E) for bright field microscopy.

### Drug Resistance Testing

The outcome of drugs was defined by the viability of B-ALL cells as a cytotoxic end point after drug treatment. B-ALL cells were cultured alone or co-cultured with niche cells in 3D hydrogel devices for 24 hr, and then incubated with 1 μM nilotinib (NIL, Cayman Chemical), 1 μM prednisone (PRE, Sigma), and 0.1 μM vincristine (VCR, Sigma) for 48 hr. The cell viability was quantitatively determined by using Calcein-AM (Thermo Fischer Scientific) and DAPI, which respectively stained the live and dead cells. Briefly, cells were rinsed thrice with PBS and incubated with Calcein-AM and DAPI (1 g/mL and 5 μg/mL in PBS or medium) solution for 30 min at 37° C., followed by a final rinse with PBS. Afterward, cells were observed under a fluorescence microscope, and cellular viability was quantitatively measured by counting the number of objects in the green (live cells) and red (dead cells) channels. Untreated groups of B-ALL cells were used as controls to benchmark the drug resistance of different groups.

### Combinational Drug Regimens

The 3D biomimetic leukemic niche model was exploited to test the combinational drug regimens in vitro. Generally, the murine leukemic niche models were cultured for 24 hr after fabrication, then respectively administrated with various pharmaceuticals [i.e. 5 μg/mL AMD3100 (AMD), 1 μg/mL BIO5192 (BIO), and 10 μM BAY 11-7082 (BAY), correspondingly], and concomitantly treated with 1 μM NIL, 1 μM PRE or 0.1 μM VCR for 48 hr. The viability of B-ALL cells was measured by the DAPI staining as described above.

scRNA-Seq

The leukemia BM niche samples (designated as B-ALL-Niche, B-ALL co-cultured with niche cells) and control groups of leukemia culture alone (B-ALL alone) and niche cells (HUVEC, hMSCs, and hFOB) culture without leukemia (Niche alone) were engineered and cultured on-chip for 7 days as described above. Single-cell suspensions of B-ALL samples were prepared by off-chip recovery with nattokinase (50 Fu/ml) (Carrion B et al., Tissue Engineering Part C: Methods. 2014 Mar. 1; 20(3): 252-63) and pre-labeled with different anti-human hashtag antibodies (TotalSeq™, Biolegend) and mixed into two samples, REH BM niche and SUP BM niche. The libraries were prepared using the Chromium Single Cell 3′ Reagent Kits (v3): Single Cell 3′ Library & Gel Bead Kit v3 (PN-1000075), Single Cell 3′ Chip Kit v3 (PN-1000073) and i7 Multiplex Kit (PN-120262) (10× Genomics), and following the Single Cell 3′ Reagent Kits (v3) User Guide (manual part no. CG000183 Rev B). Libraries were then run on an Illumina NovaSeq 6000 using 28 bp read 1, 8 bp i7 index, and 91 bp read 2.

scRNA-Seq Data Pre-Processing

Sequencing results were demultiplexed and converted to FASTQ format using Illumina bcl2fastq software. The Cell Ranger Single-Cell Software Suite was used to perform sample demultiplexing, barcode processing, and single-cell 3′ gene counting. The cDNA insert was aligned to the hg38/GRCh38 reference genome. Only confidently mapped, nonPCR duplicates with valid barcodes and unique molecular identifiers (UMIs) were used to generate the gene-barcode matrix. Further analysis including the identification of highly variable genes, dimensionality reduction, standard unsupervised clustering algorithms, and the discovery of differentially expressed genes was performed using the Seurat R package (https://github.com/satijalab/seurat).

The leukemia BM niche samples (designated as B-ALL-Niche) were prepared in parallel with control groups of leukemia culture alone (designated as B-ALL alone) and niche cells (HUVEC, hMSCs, and hFOB) culture without leukemia (designated as Niche alone). For cell hashing, cells retrieved from groups of B-ALL alone, Niche alone, and B-ALL-Niche were respectively labelled with three hashtag antibodies (TotalSeq™, BioLegend), i.e. hashtag-1: GTCAACTCTTTAGCG (SEQ ID NO.: 1); hashtag-2: TGATGGCTATTGGG (SEQ ID NO.: 2); hashtag-3: TTCCGCCTCTCTTTG (SEQ ID NO.: 3). Following, hashtag oligo (HTO) counts were quantified using CITE-seq-count. RNA counts for each cell were preprocessed as described above.

To exclude low quality cells as well as cells that were extreme outliers in terms of library complexity and may possibly include multiple cells or doublets, the distribution of genes detected per cell was calculated and cells in the top and bottom 2% quantiles within each sequencing library were removed. Cells with more than 10% of the transcripts coming from mitochondrial genes were additionally removed. The data was normalized by the total expression, multiplied this by a scale factor of 10,000 and log-transformed. HTOs for each cell were normalized using a centered log ratio (CLR) transformation across cells and demultiplexed using the HTODemux function in Seurat. Cell doublets and background empty droplets identified based on HTO values were removed.

### Visualization and Clustering

To visualize the data, the dimensionality of the scaled integrated data matrix was further reduced to project the cells in two-dimensional space using PCA followed by uniform manifold approximation and projection (UMAP) (https://umap-learn.readthedocs.io/) based on 40 PCs with 30 nearest neighbors used to define the local neighborhood size with a minimum distance of 0.3 for the datasets. The resulting PCs were also used as a basis for partitioning the dataset into clusters using a smart local moving (SLM) community detection algorithm (https://www.ludowaltman.nl/slm/) using 30 nearest neighbors for the datasets. A range of resolutions (0.1-10) was utilized to establish a sufficient number of clusters to separate known populations based on the expression of established markers.

To find markers that define individual clusters, pairwise differential expression analysis was performed using the Wilcoxon rank sum test with Bonferroni multiple-comparison correction for each cluster against all other clusters for genes that were detected in at least 10% of the cluster cells, keeping the genes that were significant in each of the comparisons (fold-change difference >10% with adjusted p-value <0.01).

### Gene Set Enrichment Analysis (GSEA)

Gene set module scores for each cell were calculated using the average expression levels of every gene signature, subtracting the aggregated expression of randomly selected control genes. To quantify the pathways altered across the different conditions, genes were ranked based on the fold-changes between them. Statistical analysis was performed using the fgsea R package (https://bioconductor.org/packages/release/bioc/html/fgsea.html) for the pre-ranked gene list based on the differential expression against the MSigDB gene sets (https://software.broadinstitute.org/gsea/msigdb) with 10,000 iterations. To determine the similarity of cell types across datasets, Gene Set Variation Analysis (GSVA) 48 was utilized to evaluate the enrichment of population-specific markers within each cluster based on the cluster averaged log-transformed expression matrix.

### Statistics

Data were first analyzed for normality and then compared with unpaired t test or one-way analysis of variance (ANOVA) using Prism8.0 (GraphPad). *p<0.05 and **p<0.01 were considered significantly different. The results, including the error bars in the graphs, were given as the mean±standard deviation (s.d.). Details are reported in each figure.

The results are now described.

### Modeling the In Vivo Leukemic BM Niche in an In Vitro Leukemia-On-a-Chip System

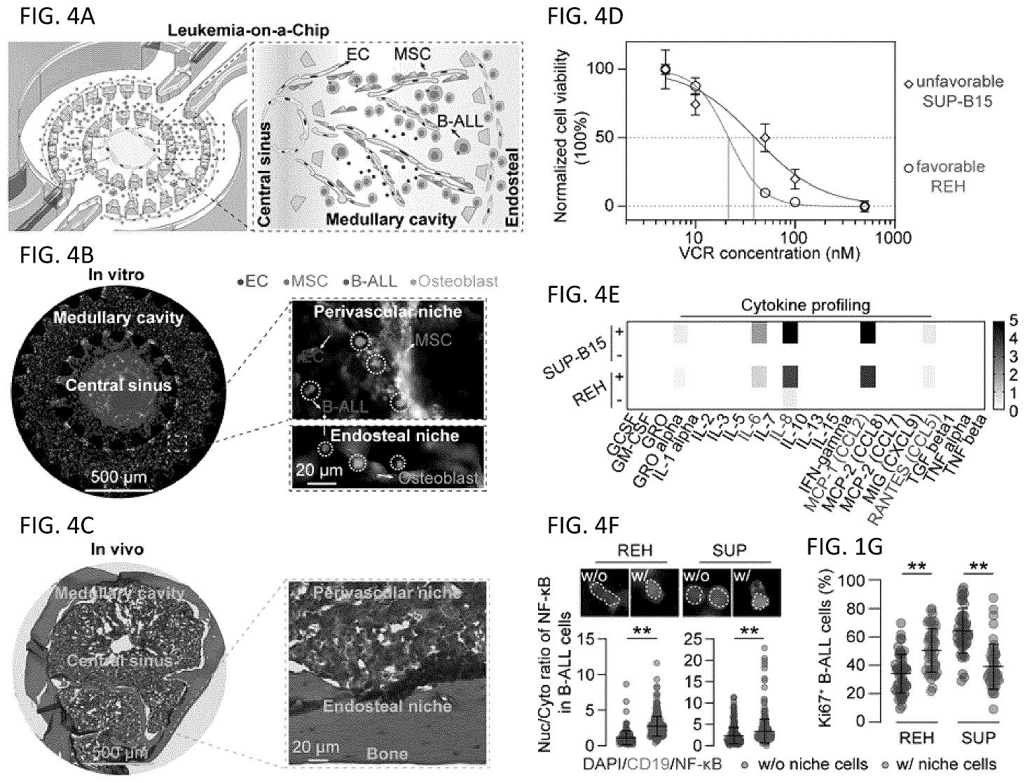

An in vitro organotypic leukemic BM microenvironment, dubbed ‘Leukemia-on-a-Chip’, was engineered to mirror the in vivo pathology of the leukemic BM niche and identify underlying mechanisms responsible for B-ALL chemo-resistance. This microfluidics-based microphysiological system integrates key features that replicate in vivo BM tissue architecture and monitors dynamic B-ALL and leukemic BM niche interactions in real time with live-cell imaging (FIG. 4A through FIG. 4G and FIG. 3A through FIG. 3G). Specifically, Leukemia-on-a-Chip was fabricated using standard soft lithography with polydimethylsiloxane (PDMS) (Ma C et al., Analytical chemistry. 2016 Feb. 2; 88(3): 1719-27; Ma C et al., Lab on a Chip. 2016; 16(14): 2609-17). Leukemia-on-a-Chip culture (FIG. 4A and FIG. 3A through FIG. 3C) was compartmentalized into a biomimetic central venous sinus (center region; red), a medullary cavity (middle ring; green), and endosteal regions (outer ring) connected with four media reservoirs for long-term media supply. These three functional regions were partitioned by regularly-spaced trapezoid micropillars that confine cell-embedded hydrogels, by balancing surface tension and capillary forces, to overall mimic the native in vivo BM tissue architecture of leukemia.

The reconstituted on-chip leukemic BM niche houses a biomimetic central venous sinus, medullary cavity, and endosteum anatomical (endosteal) regions (FIG. 4B and FIG. 3D through FIG. 3F) that permit spatially-defined, intercellular communication (i.e. B-ALL, ECs, MSCs, and osteoblasts) to interrogate cytokine and adhesive signaling milieus in conferring B-ALL chemo-resistance. In parallel, the on-chip reconstruction of the B-ALL BM niche was compared to the in vivo BM tissue architecture of recipient mice injected with leukemic blasts, specifically using a high-risk Ph+ B-ALL pre-clinical C57BL/6 mouse model (FIG. 4C) (Li S et al., The Journal of experimental medicine. 1999 May 3; 189(9): 1399-412). The results demonstrate Leukemia-on-a-Chip resembles the in vivo spatial architecture and cellular composition of the leukemia BM tissue.

### Mapping the Leukemia Niche Heterogeneity Across Molecularly-Distinct B-ALL Subtypes

Notably, interpreting the liaison between tumor heterogeneity and therapeutic response, such as ETV6-RUNX1⁺ B-ALL patients are associated with favorable outcome, whilst Ph+ B-ALL patients display poor responses to conventional agents, as compared to tyrosine kinase inhibitor (e.g. nilotinib), is still an outstanding issue (Churchman M L et al., Cancer cell. 2015 Sep. 14; 28(3): 343-56). Moreover, due to the technical difficulties associated with isolating human BM stromal subpopulations in primary leukemic patient BM samples, the evolving interactions between human BM microenvironment and leukemia remain unclear. To study the heterogeneity in B-ALL human BM microenvironments, the Leukemia-on-a-Chip platform was utilized to establish human B-ALL BM niche in vitro models by seeding either ETV6-RUNX1⁺ REH (ATCC) and Ph+ SUP-B15 (SUP, ATCC) human B-ALL cell lines with a combination of human umbilical vein endothelial cells (HUVEC, Lonza), human mesenchymal stem cells (hMSCs, Lonza), and human osteoblasts (hFOB 1.19, ATCC) that aimed to mimic components of the human BM niche. Notably, REH and SUP BM niches showed distinct chemotherapy sensitivity in the biomimetic devices upon exposure to increasing doses of vincristine (VCR, Sigma), with SUP B-ALL co-cultured with niche cells showing more resistant to VCR than REH co-cultured with niche cells (FIG. 4D), consistent with insensitivity of Ph+ B-ALL to conventional chemotherapeutic agents. To understand the differences in chemo-sensitivity that exists between human B-ALL cell lines, SUP and REH, co-cultured with BM niche cells, differences were quantified in cytokines present in the supernatant of these respective devices. Here it is shown that progressive production of CCL2, CCL5, IL-6 and IL-8 were observed upon seeding and growth of either REH or SUP in the leukemia BM niche model and that SUP BM niche had a slightly higher production of CCL2, IL6 and IL-8, as compared to REH BM niche (FIG. 4E). NF-κB signaling was also observed to be enhanced in both leukemia subtypes upon co-culture with niche cells (FIG. 4F), based on the nuclear/cytoplasmic expression of phosphorylated p65 subunit, a subunit of NF-κB. Moreover, SUP B-ALL demonstrated a decreased percentage of Ki67 staining, whereas REH B-ALL showed an increased Ki67 expression, compared between with and without co-culture with niche cells (FIG. 4G).

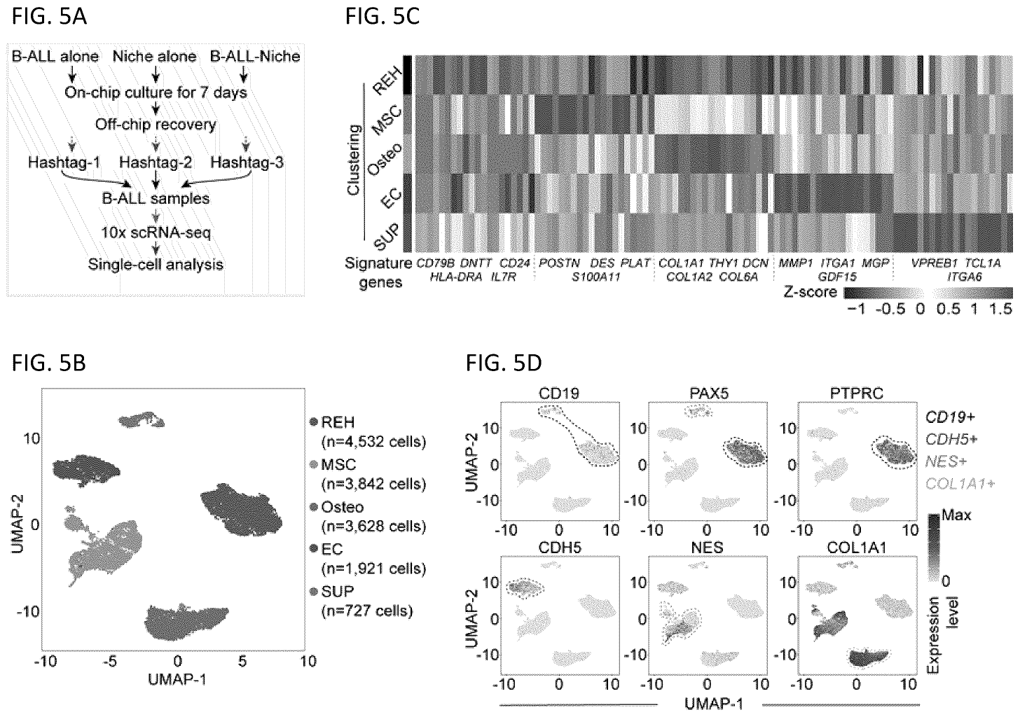

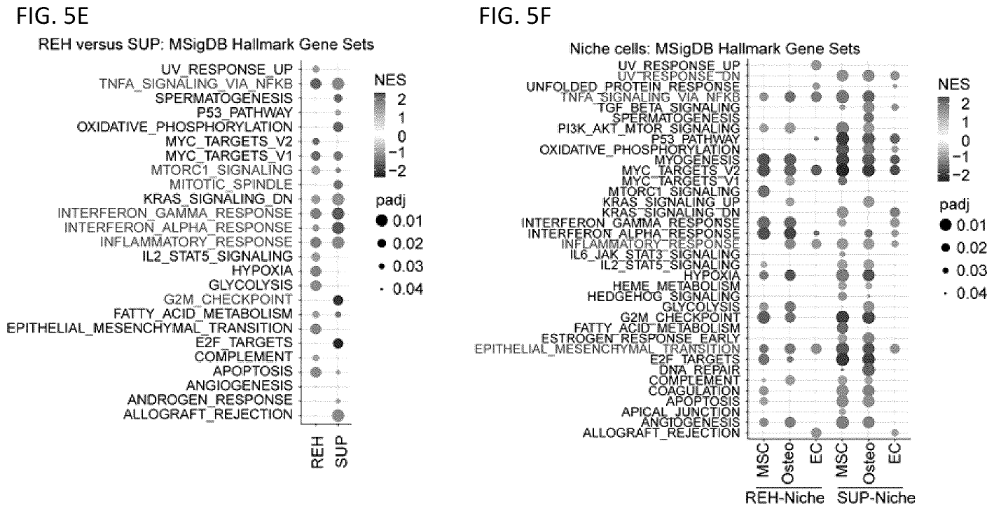

To further elucidate such heterogeneity across genetically-distinct human B-ALL blasts and their related BM niches, the powerful scRNA-seq analysis tool was leveraged (characterizing the BM microenvironment with limited cell input number (Witkowski M T et al., Cancer Cell. 2020 Jun. 8; 37(6): 867-82; Tikhonova A N et al., Nature. 2019 May; 569(7755): 222-8) to molecularly explore the favorable (REH) and unfavorable (SUP) human leukemia BM niches. Single-cell suspensions were prepared and sequenced for the engineered REH and SUP BM niche samples, as well as control groups (i.e. B-ALL alone and Niche alone) (FIG. 5A). In total, data for 14,650 cells was obtained (FIG. 5B). Based on the experimental design of input and culturing five distinct cell types (SUP B-ALL, REH B-ALL, osteoblasts, HUVECs and hMSCs), single-cells were classified into five distinct clusters (FIG. 5B, FIG. 5C) that distinctively expressed (REH and SUP B-ALL) CD19⁺PAX5⁺, (hMSC) NES⁺, (Osteoblasts) COL1A1⁺, and (HUVECs) CDH5⁺ (FIG. 5D). To further validate cell type identity of the niche cell clusters, the gene signatures of the three niche cell clusters were compared with previously described gene of murine BM niche cells (Tikhonova A N et al., Nature. 2019 May; 569(7755): 222-8). Together, these results confirmed that conserved gene signatures exist between previously-described murine niche cell types and gene expression profiles of hMSC, osteoblasts and HUVECs derived from the devices (FIG. 6A).

Gene sets enrichment analysis was performed of Molecular Signature Database (MSigDB) Hallmark gene sets for all the cell types from the two leukemia niche samples (FIG. 5E, FIG. 5F). Significantly-enriched gene expression profiles related to TNFA signaling via NF-κB and inflammation response activation were associated with most of the cell types, highlighting the inflammation situation upon leukemia progression. Of particular interest, SUP B-ALL, compared to REH B-ALL, significantly decreased expression of mitotic spindle and G2M checkpoint related gene sets, which indicated that unfavorable B-ALL may demonstrate much more quiescence in comparison with favorable B-ALL in the leukemic BM niche (FIG. 5E). In contrast, niche cells from both leukemia niches augmented expression of epithelial mesenchymal transition related gene sets, suggestive of leukemia-driven niche remodeling (FIG. 5F).

Having established gene expression profiles for the BM nice cells and leukemic blasts present in the in vitro devices, the cell type-specific and shared signaling pathways that exist throughout the leukemic microenvironments were next determined. Specially, the mRNA expression of leukemia survival and proliferation/quiescence regulators was assessed, such as NFKB1A and MKI67 as well as niche-derived cytokine and adhesive signaling throughout all five clusters (FIG. 5A through FIG. 5F and FIG. 6B through FIG. 6D). In the presence of niche cells, for instance, it was observed that SUP B-ALL blasts slightly decreased MKI67 mRNA expression relative to those in the absence of niche cells, which is in consistent with on-chip observance (FIG. 4G and FIG. 6B). In addition, SUP B-ALL cells increased NFKB1A expression when cultured with niche cells, whereas this was not the case for REH B-ALL. To clarify the variance of NFKB1A expression, gene set enrichment analysis of NF-κB pathways was applied on these two B-ALL cell types (FIG. 6C). The results showed both B-ALL blasts were more enriched with NF-κB related signaling when cultured in leukemia BM niche models. As for gene expression of adhesive signaling in niche cells, hMSCs from the SUP BM niche showed increased expression of OPN, while hMSCs from the REH BM niche showed decreased OPN expression, implicating such heterogeneity may contribute to diverse extent of quiescence in the two leukemia subtypes (FIG. 6D), though the protein levels may not fully correlate with the mRNA levels observed. Therefore, together with scRNA-seq, cytokine secretion and immunofluorescence analysis, a comprehensive molecular map of the engineered human BM niche was generated for the two representative B-ALL cell types, which may provide a landscape to systematically interrogate the disease progression of varying leukemia subtypes and the accompanying transformation of non-malignant BM niche components.

### Monitoring the Temporal Dynamics of B-ALL Progression in the Leukemic Niche

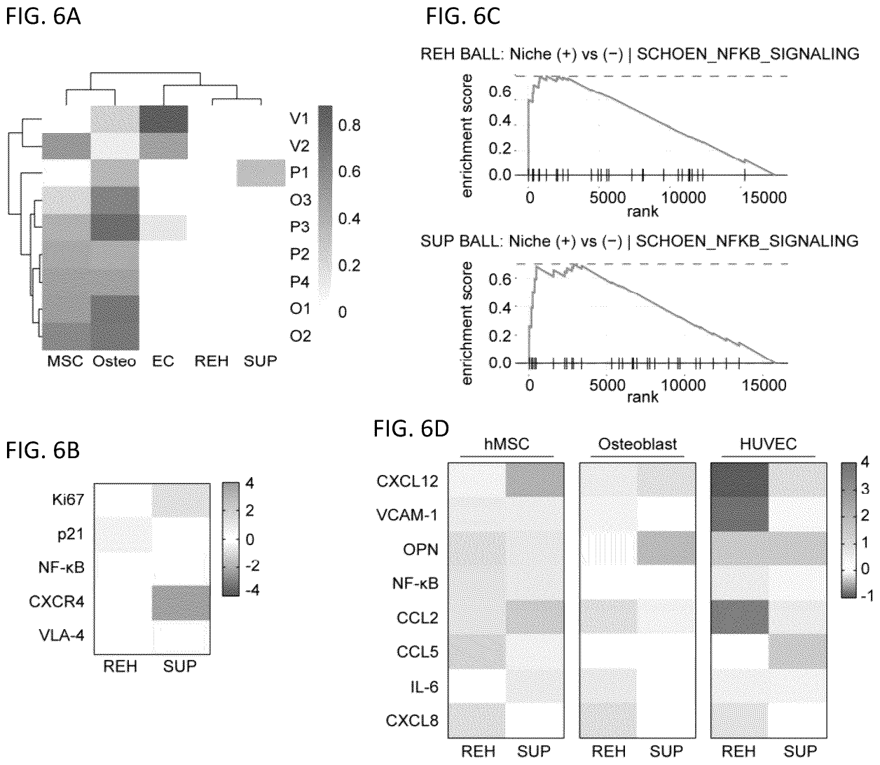

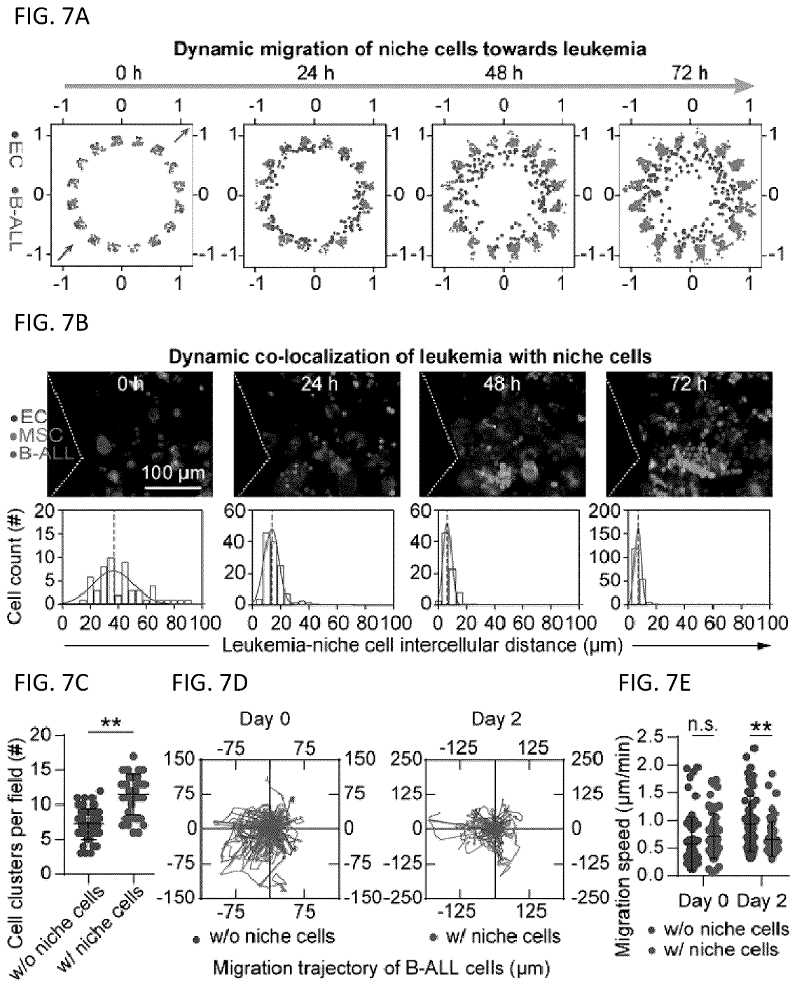

To mechanistically understand the dynamic microenvironmental interactions during leukemic pathogenesis, the in situ migratory patterns of murine Ph+ B-ALL and niche cells (containing ECs and MSCs) were longitudinally monitored for a three-day period with time-lapse imaging. To clearly visualize and distinguish Ph+ GFP+ leukemia cells and niche cells, ECs were labeled with CellTracker Red CMTPX dye (Thermo Fischer Scientific). The migration of niche cells towards B-ALL cells were first characterized by intentionally segregating niche cells into the ring area and B-ALL cells in the central region and the dynamic migration of B-ALL cells and niche cells (especially ECs) were mapped at the interface of central and ring regions. B-ALL cells attracted ECs during the three-day culture, as indicated by the presence of ECs in the central area (FIG. 7A). In addition, the intercellular distance was quantified between B-ALL and niche cells during co-culture, and the results demonstrated B-ALL and niche cells dynamically co-localized (FIG. 7B). An enhanced clustering of B-ALL cells was observed around niche cells comparing to the B-ALL mono-culture condition, indicating that niche cells may provide additional adhesive sites to facilitate B-ALL clustering (FIG. 7C). Time-lapse migration analysis of B-ALL cells revealed that leukemic cells either cultured in the presence or absence of niche cells were comparably motile at the culture onset (day 0), whereas after a two-day culture, leukemia blasts co-cultured with niche cells were less motile, as compared to those cultured in the absence of niche cells (FIG. 7D, FIG. 7E), demonstrating the temporally evolved features in chemotactic and adhesive signaling among B-ALL blasts and niche cells. Together, these studies prove in vivo B-ALL BM pathology is faithfully recapitulated in the in vitro Leukemia-on-a-Chip model that permits real-time and longitudinal monitoring of the in situ temporal microenvironmental evolution during leukemic pathogenesis, revealing unique migration patterns and clustering behaviors of B-ALL blasts in the leukemic BM niche.

### Dissecting the Niche-Derived Signal Underlying Leukemia Progression

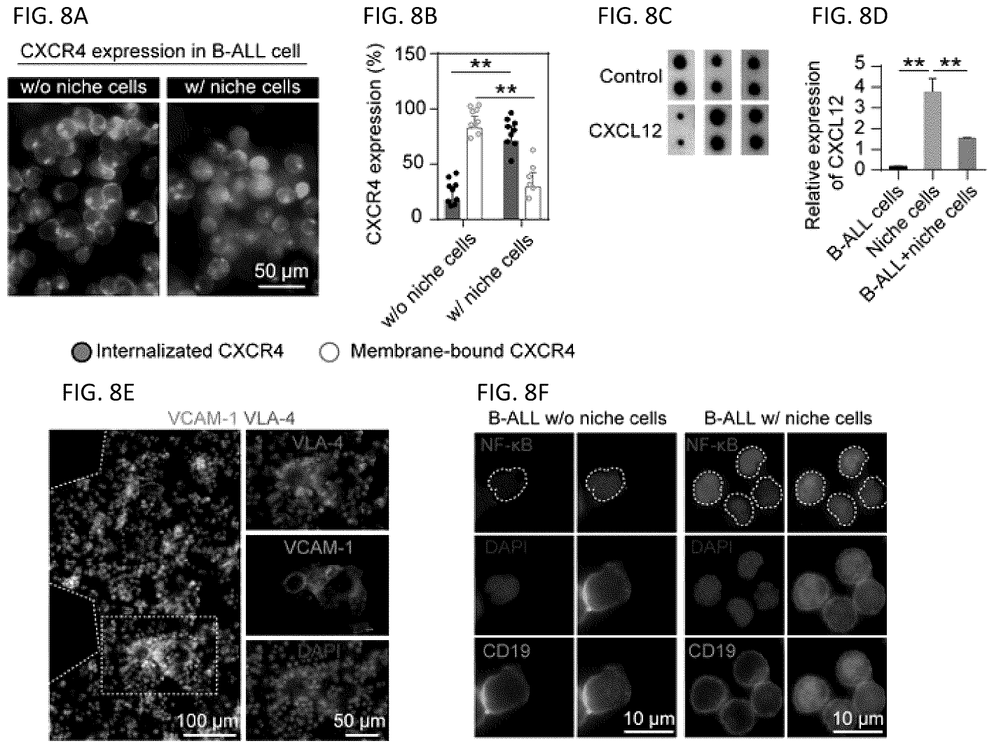

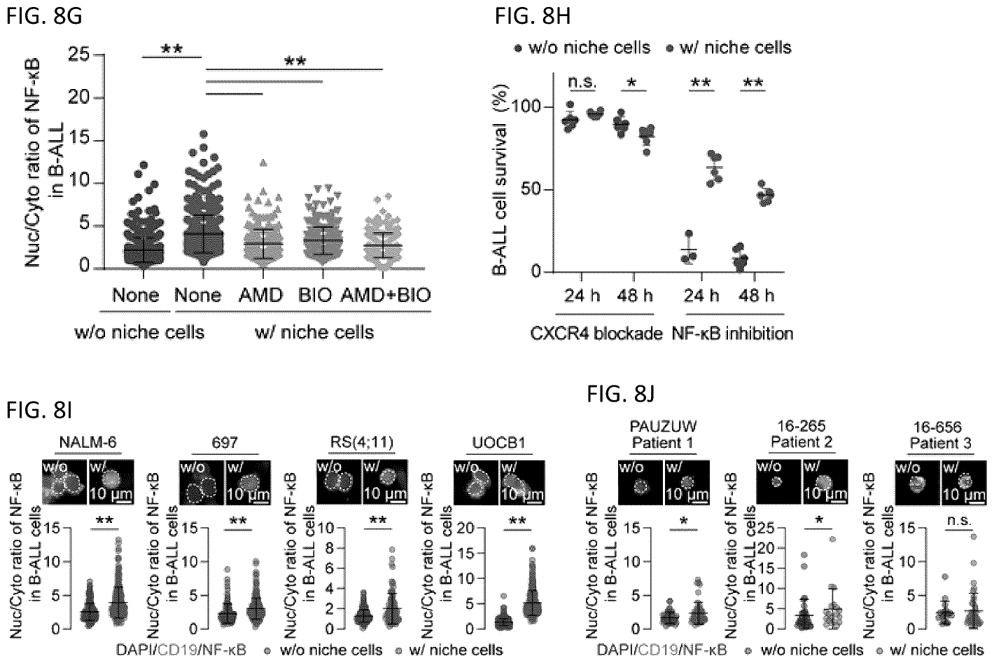

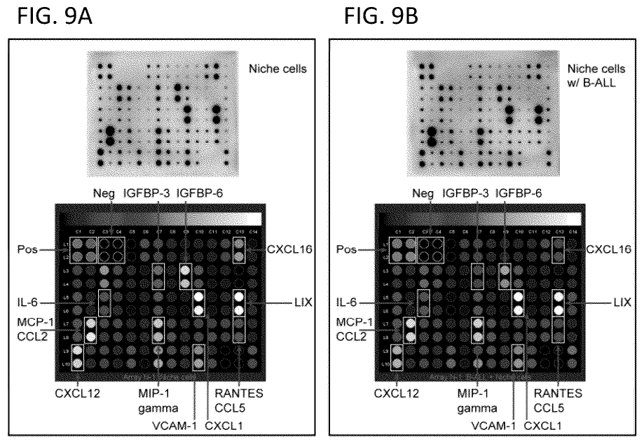

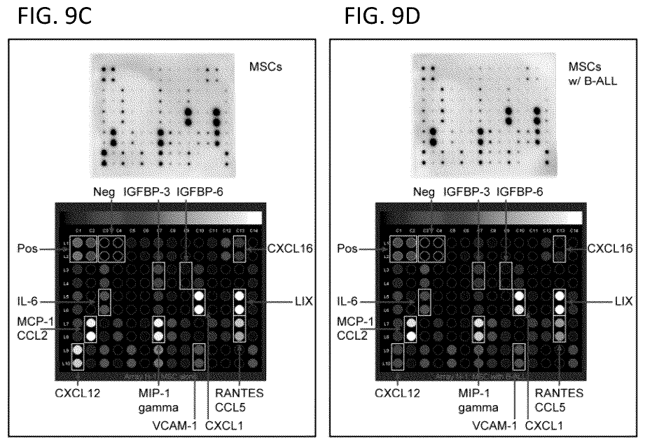

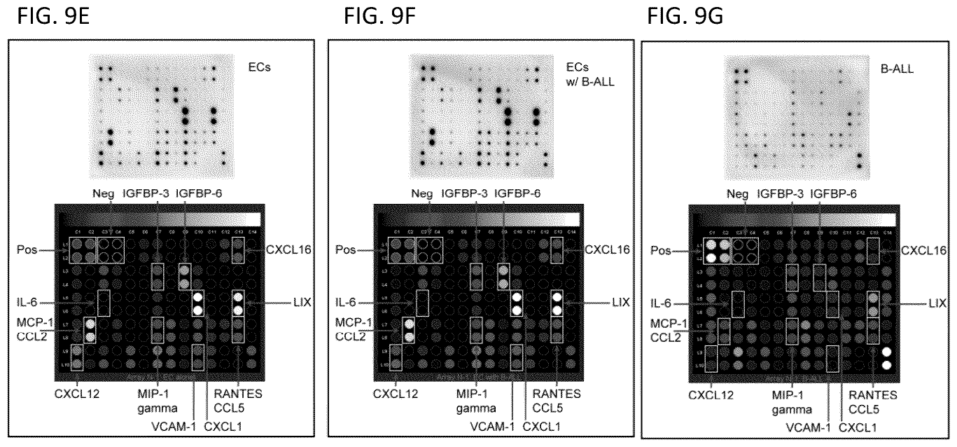

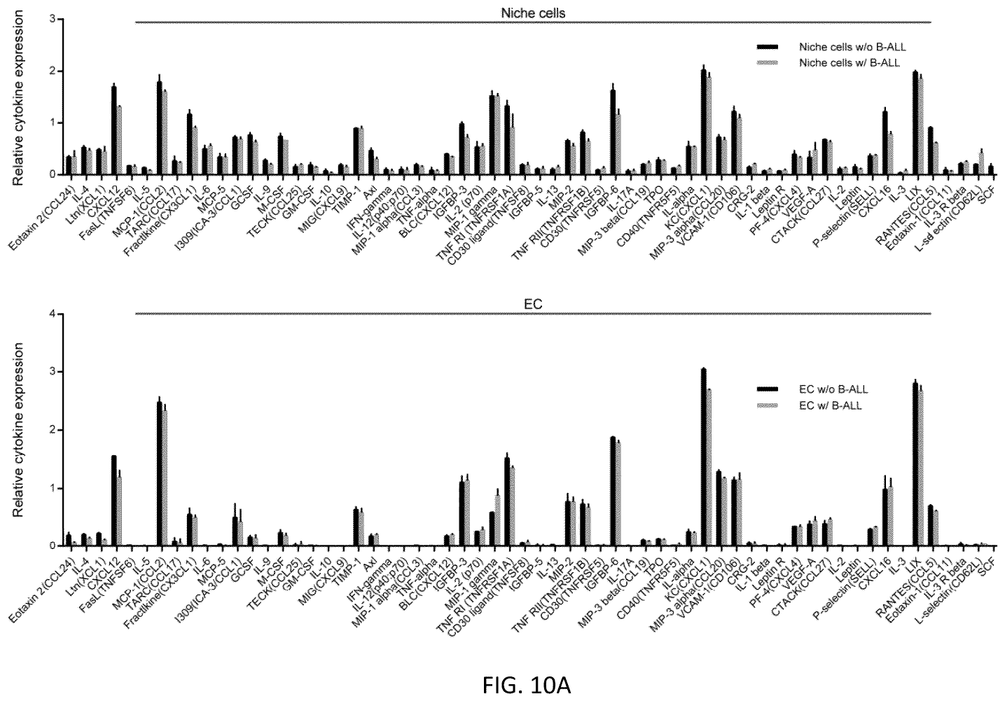

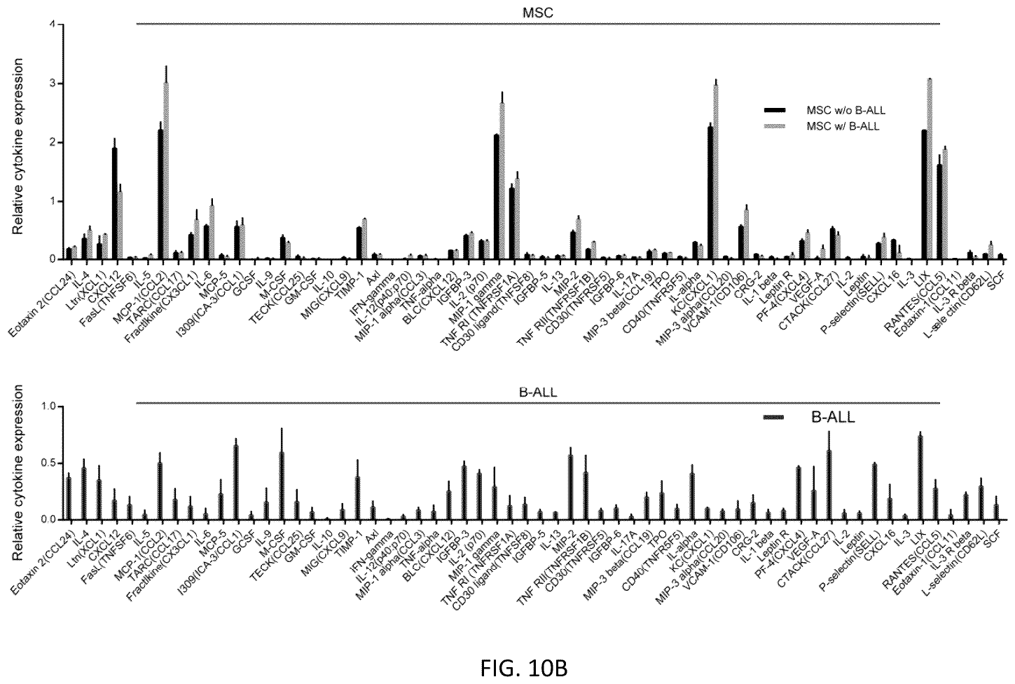

It was previously revealed in vivo T-ALL and B-ALL studies that CXCL12 from BM niche may induce leukemia progression via its receptor CXCR4, thus supporting leukemia survival (Passaro D et al., Cancer cell. 2015 Jun. 8; 27(6): 769-79; Pitt L A et al., Cancer cell. 2015 Jun. 8; 27(6): 755-68; Sison E A et al., Oncotarget. 2014 October; 5(19): 8947). To corroborate the results of scRNA-seq analysis of engineered leukemic BM niche, the identified signaling was further studied in the in vitro Leukemia-on-a-Chip model. CXCR4+ B-ALL cells were co-localized with CXCL12⁺ niche cells in the leukemic BM niche (FIG. 7G), confirming the potent role and the existence of spatiotemporal regulation of niche cell-derived signaling in the evolving progression of B-ALL. Steady-state cell surface expression of CXCR4 results from a balance between endocytosis, intracellular trafficking, and recycling (English E J et al., Journal of Biological Chemistry. 2018 Jul. 20; 293(29): 11470-80). By quantifying the sub-cellular CXCR4 (Abcam, ab124824) receptor distribution in B-ALL cells, B-ALL cells in the B-ALL BM niche tended to have a higher ratio of CXCR4 internalized within the cytoplasm than those without niche cells, indicating active leukemia-intrinsic downstream events by CXCL12/CXCR4 signaling from interactions with the leukemic BM niche (FIG. 8A, FIG. 8B). To confirm the source of CXCL12, a membrane-based enzyme-linked immunosorbent assay (ELISA) cytokine analysis was performed on niche cells (FIG. 8C, FIG. 8D, FIG. 9A through FIG. 9G, and FIG. 10A and FIG. 10B). The results showed that niche cells (without culture with B-ALL blasts) exhibited a high production of CXCL12, while decreased within the leukemic niche upon 2-day co-culture with B-ALL. This indicates CXCL12 may function in the early stage of leukemia progression, such as induction of leukemia migration (FIG. 7D, FIG. 7E). The overall cytokine profiles also demonstrated that niche cells secreted an array of cytokines, such as CCL2 and CCL5, regulating leukemia progression (FIG. 9A through FIG. 9G and FIG. 10A and FIG. 10B), which is consistent with human system (FIG. 3E) and previous in vivo studies (Jacamo R et al., Blood. 2014 Apr. 24; 123(17): 2691-702; de Rooij B et al., haematologica. 2017 October; 102(10): e389).

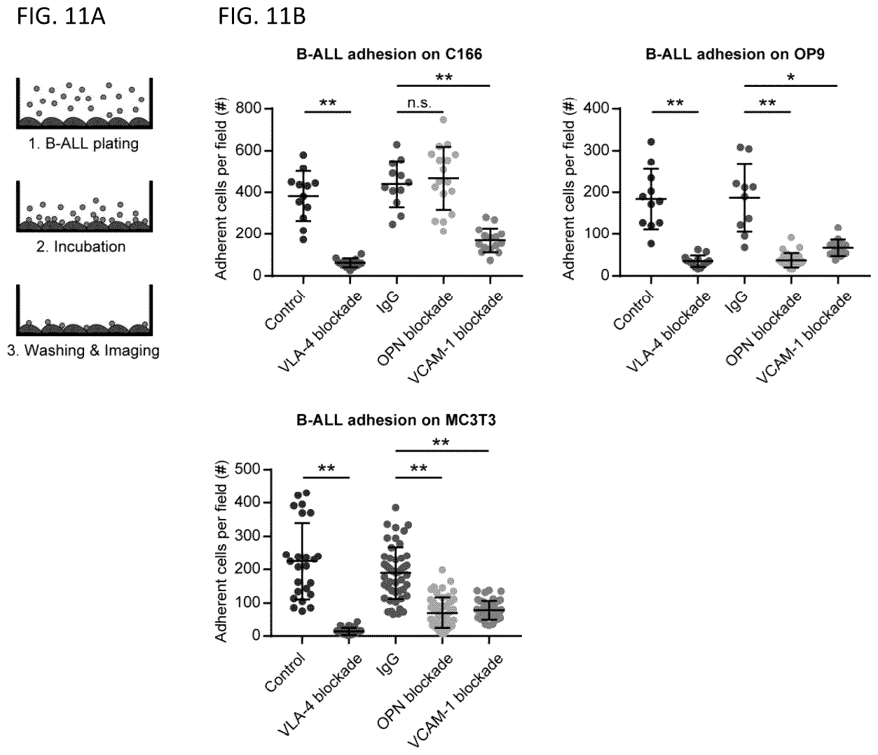

Leukemic blasts were observed to physically cluster around niche cells, indicating that niche cells may provide unique adhesion sites for engrafting B-ALL cells (FIG. 7C). HSC retention within the BM microenvironment is mediated by VLA-4/VCAM-1 signaling; similarly, leukemic blasts have been shown to engage perivascular stromal cells via this intercellular adhesive signaling (Boyerinas B et al., Blood, The Journal of the American Society of Hematology. 2013 Jun. 13; 121(24): 4821-31; Jacamo R et al., Blood. 2014 Apr. 24; 123(17): 2691-702; Hsieh Y T et al., Blood, The Journal of the American Society of Hematology. 2013 Mar. 7; 121(10): 1814-8). To confirm intercellular adhesive interactions between B-ALL cells and niche cells, VLA-4+ B-ALL cells (Abcam, ab202969) were found to co-localize with VCAM-1⁺ niche cells in on-chip cultures (FIG. 8E). In addition, it was confirmed that blocking VLA-4 with BIO 5192 (BIO, R&D Systems) can significantly decrease the intercellular adhesion of B-ALL cells onto niche cells (FIG. 11A and FIG. 111B), proving that the niche cell-mediated VCAM-1/VLA-4 signaling is prominently involved in regulating B-ALL adhesion and clustering (FIG. 8E).

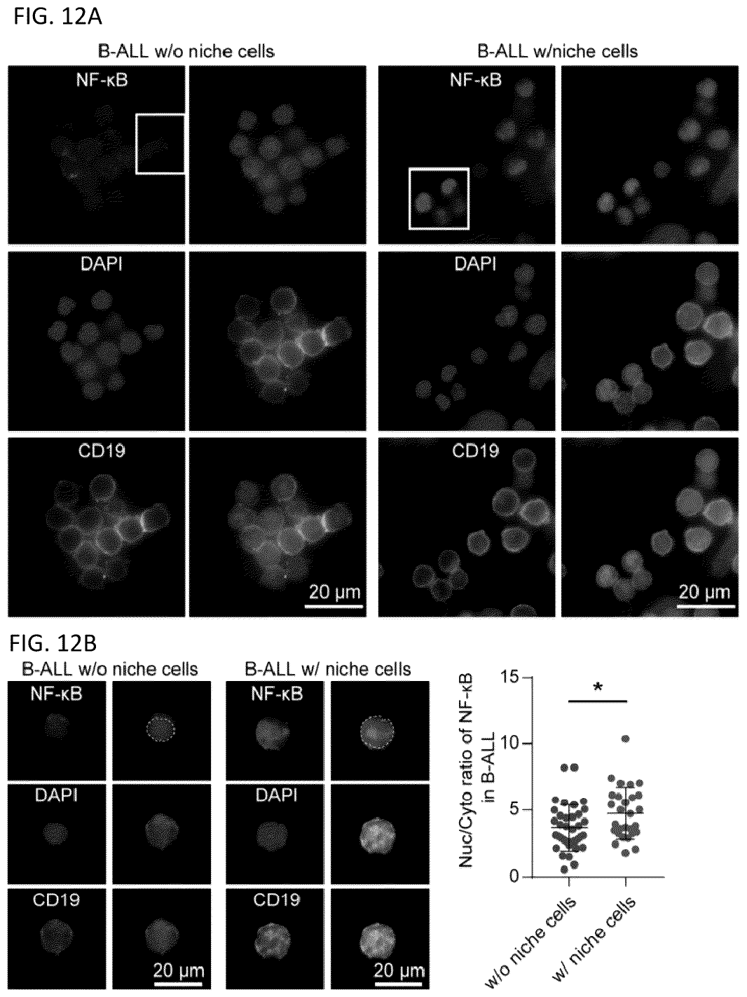

It remains not fully defined that how these cytokine and adhesive signaling regulate B-ALL progression and chemo-resistance. Previous evidence indicates that CXCR4 internalization and subsequent activation of phosphatidylinositol-3-OH kinase and Akt kinase lead to upregulated NF-κB pro-survival signaling in various cancers (English E J et al., Journal of Biological Chemistry. 2018 Jul. 20; 293(29): 11470-80). Together with CXCL12/CXCR4 signaling, direct cell-cell contact via VCAM-1/VLA-4 interactions may also be involved in enhancing leukemia survival by activating NF-κB signaling in leukemia as revealed from the scRNA-seq analysis results and previous studies (Boyerinas B et al., Blood, The Journal of the American Society of Hematology. 2013 Jun. 13; 121(24): 4821-31; Jacamo R et al., Blood. 2014 Apr. 24; 123(17): 2691-702; Hsieh Y T et al., Blood, The Journal of the American Society of Hematology. 2013 Mar. 7; 121(10): 1814-8). To determine whether CXCL12 cytokine and VCAM-1/VLA-4 adhesive signaling axes enhance B-ALL survival via regulating downstream NF-κB signaling, the measured levels of NF-κB activation present in B-ALL cells cultured with or without niche cells were compared using the Leukemia-on-a-Chip system as well as 2D culture (FIG. 8F, FIG. 12A, and FIG. 12B). BM niche cells significantly enhanced NF-κB nuclear translocation in B-ALL cells, as compared to those cultured in the absence of niche cells, which is consistent with observations in the engineered human system (FIG. 3F). It was also determined that blocking both CXCL12/CXCR4 and VCAM-1/VLA-4 signaling axes using either CXCR4 inhibitor AMD3100 (AMD, Sigma) or VLA-4 inhibitor BIO suppressed the nuclear translocation of NF-κB in B-ALL cells (FIG. 8G). To further understand the importance of the niche-derived NF-κB signaling in B-ALL survival, B-ALL cell viability was assayed and compared in the presence or absence of niche cells when blocking the NF-κB signaling with its inhibitor, BAY 11-7082 (BAY; EMD Millipore). Blocking NF-κB signaling in B-ALL cells significantly decreased B-ALL survival in the absence of niche cells, whereas co-culture with niche cells partially rescued B-ALL cell survival from BAY treatment (FIG. 8H). These results together prove that cytokine signaling (CXCL12/CXCR4 axis) and adhesive signaling (VCAM-1/VLA-4 axis) between niche and B-ALL blasts may act to regulate downstream NF-κB signaling to promote B-ALL cell survival in the in vitro leukemic BM niche model.

The Leukemia-on-a-Chip was applied for testing another four genetically different types of human B-ALL leukemia cell lines [i.e. NALM-6, 697, RS(4; 11), and UOCB1], as well as three patient-derived samples (i.e. Ph+ PAUZUW, Ph+ 16-265, and non-Ph+ 16-656). The results showed that after co-culture with niche cells, NF-κB expression was significantly increased in human B-ALL cell lines (FIG. 8I) and also in Ph+ PAUZUW and 16-265 patient-derived B-ALL blasts, though it was not the case for non-Ph+ 16-656 patient blasts (FIG. 8J). Taken together, these results demonstrated the enhanced NF-κB signaling as a general pro-survival mechanism contributed by niche cells.

### Revealing the Cellular and Signaling Heterogeneity in the Leukemic BM Niche

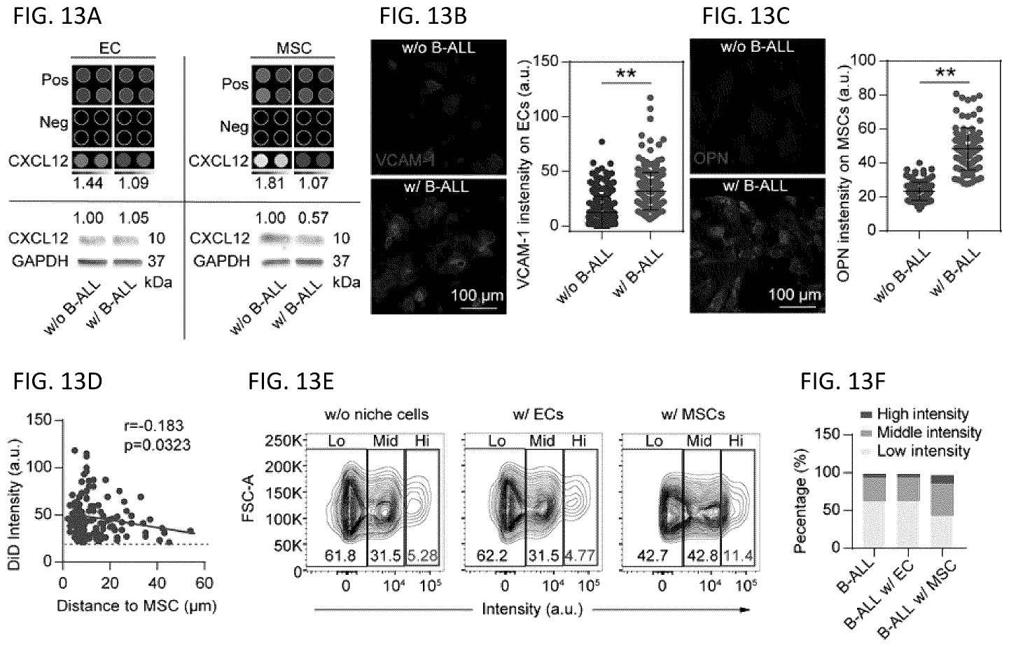

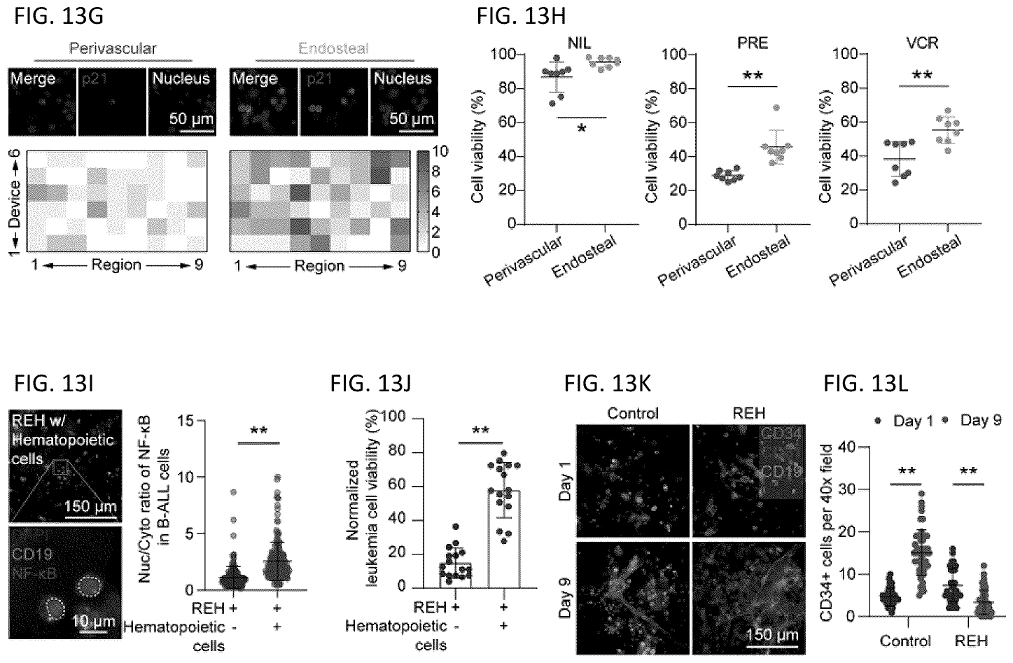

From the cytokine analysis, niche cells (ECs and MSCs) decreased CXCL12 secretion after prolonged co-culture with B-ALL cells (FIG. 8C, FIG. 8D). To understand whether murine ECs or MSCs or both cell types decrease CXCL12 secretion, ECs and MSCs were cultured, respectively, with B-ALL blasts for 2 days, and cytokine secretion profiles were monitored using membrane-based ELISA and Western blotting assays. ECs and MSCs differentially respond to the presence of B-ALL, whereas MSCs were more strongly affected by B-ALL blasts and had a reduced secretion of CXCL12 than ECs did (FIG. 13A, FIG. 9A through FIG. 9G, and FIG. 10A and FIG. 10B).

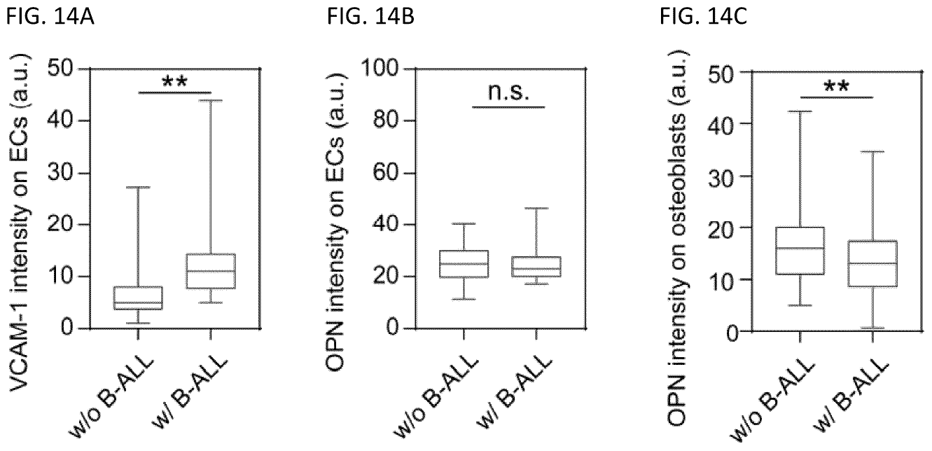

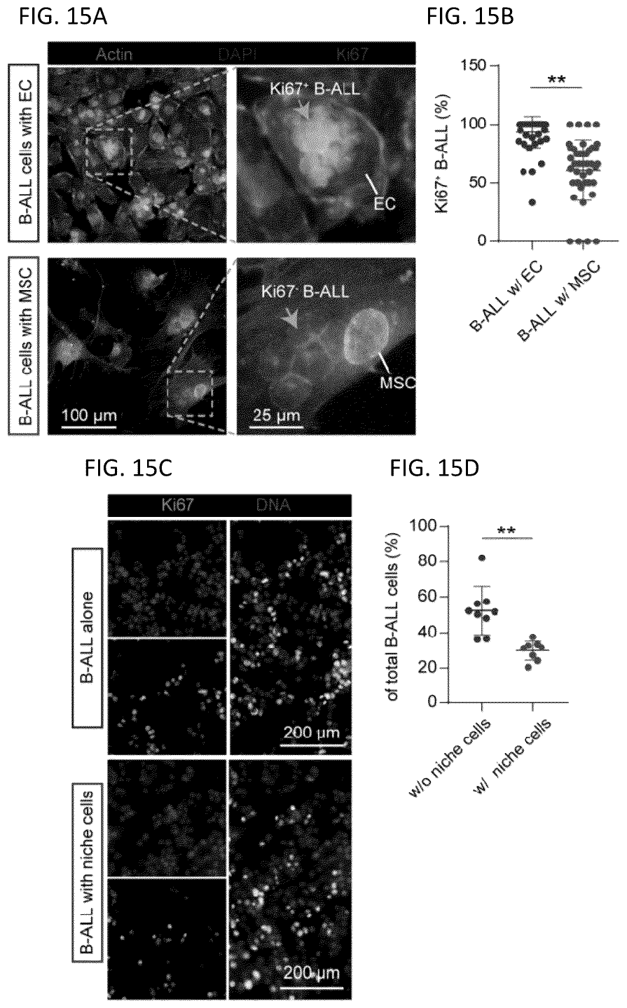

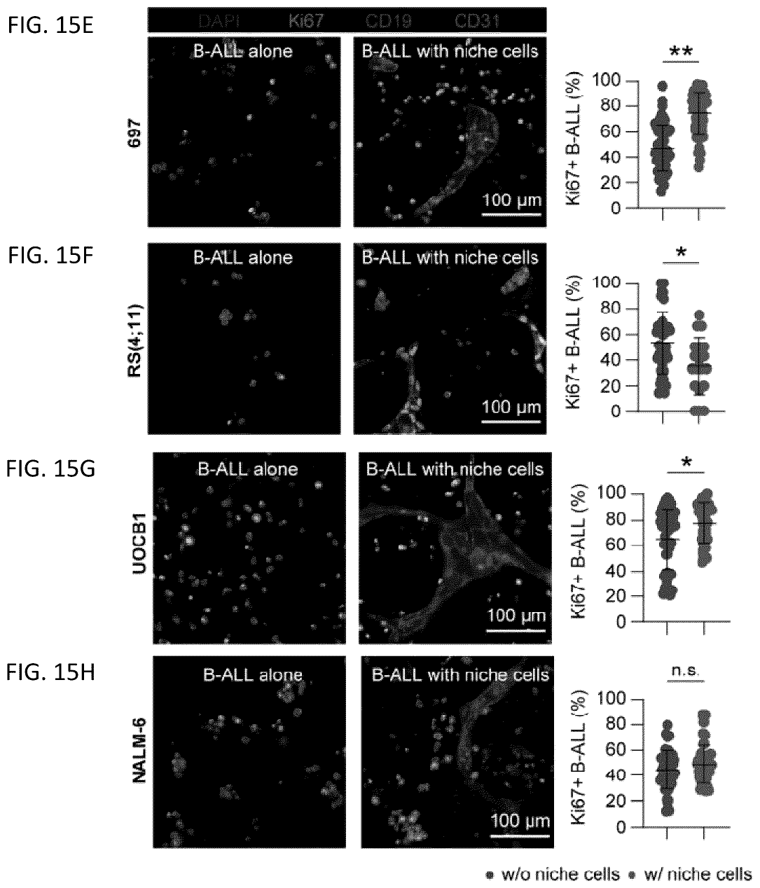

Next studied was whether ECs and MSCs all engaged in intercellular adhesive signaling, such as VCAM-1 or OPN to promote B-ALL cell adhesion. Interestingly, it was observed that a significant increase of VCAM-1 expression but no significant change of OPN expression in ECs when co-cultured with B-ALL cells (FIG. 13B and FIG. 14A, FIG. 14B). By contrast, co-culture with B-ALL elicited a significant increase of OPN expression in MSCs (FIG. 13C). Moreover, OPN blockade can significantly decrease B-ALL adhesion onto MSCs, which was absent in ECs (FIG. 12A and FIG. 12B). By staining cells with proliferation marker, Ki67 (BioLegend, 652402), a higher percentage of B-ALL cells were demonstrated to interact with MSCs were Ki67 negative, when compared to B-ALL interacting with ECs, demonstrating that MSCs may promote B-ALL dormancy (FIG. 15A, FIG. 15B). Ki67 expression was assayed in murine B-ALL and human leukemia cell lines on-chip cultured with and without niche cells, which demonstrated the heterogeneity across different types of B-ALL blasts (FIG. 15C through FIG. 15H). To study this further, B-ALL cells were labeled with the lipophilic dye, DiD, which is retained in dormant or slow-cycling cells in culture (Ebinger S et al., Cancer cell. 2016 Dec. 12; 30(6): 849-62), and then co-cultured with MSCs at a 1:1 ratio. It was found that B-ALL cells more physically close to MSCs tended to retain a higher intensity of DiD labeling, indicating MSCs critically induced B-ALL dormancy (FIG. 13D). This finding was also confirmed by flow cytometric analysis (BD Symphony™ A5 Cytometer) with GFP as a gate way. The obtained result was analyzed using FlowJo (TreeStar, BD Biosciences), which showed a greater percentage of high DiD-retaining cells in B-ALL cells co-cultured with MSCs, compared to B-ALL cells cultured in isolation or with ECs (FIG. 13E, FIG. 13F).

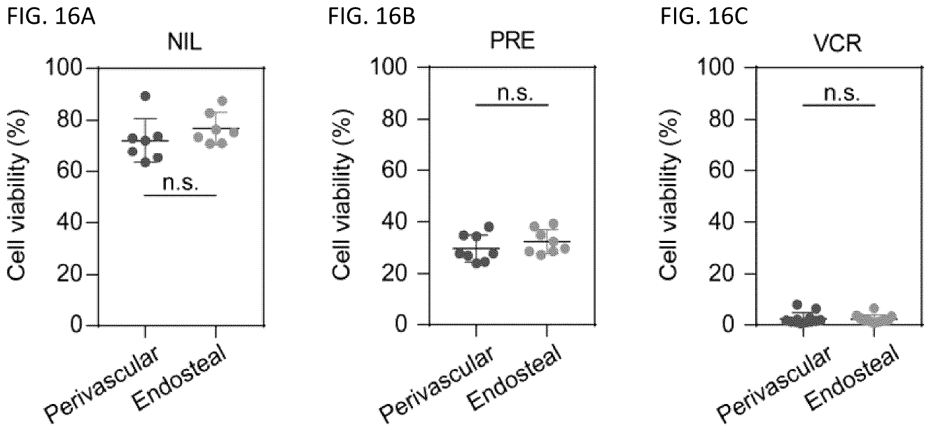

Historically, HSCs residing in either endosteal or medullary space (the peri-/vascular niche) show distinct niche-regulated cell fates (e.g. maintenance, proliferation, quiescence, and differentiation) (Ding L et al., Nature. 2013 March; 495(7440): 231-5). To further expand understanding of how perivascular and endosteal niche cells differentially regulate leukemia progression, murine MC3T3 osteoblasts were co-seeded with B-ALL cells at 1:1 ratio in the outer ring area to encircle the medullary cavity, replicating the in vivo endosteal region (FIG. 3A and FIG. 7A through FIG. 7E). First, the adhesive signaling was characterized in osteoblasts in the presence or absence of B-ALL cells. The presence of B-ALL cells slightly reduced mature osteoblast marker OPN expression in osteoblasts (FIG. 14C), indicating leukemia progression may inhibit osteoblast differentiation and maturation and be related to bone fracture/loss. Moreover, the dormant status of B-ALL cell located in the perivascular and endosteal niches were compared by characterizing its expression of p21 (Thermo Fisher Scientific, MA5-31479), a cyclin-dependent kinase inhibitor mediating cell cycle arrest (Abbas T et al., Nature Reviews Cancer. 2009 June; 9(6): 400-14). B-ALL cells in the endosteal niche had a higher number of p21-positive cells, in contrast to those in the perivascular niche (FIG. 13G). In parallel, the viability of B-ALL cells was compared in the two niches in response to 2-day treatment of three different drugs, including glucocorticoids, prednisone (PRE); microtubule inhibitor, vincristine (VCR), and targeted agent capable of BCR-ABL1 inhibition, nilotinib (NIL) (FIG. 13H and FIG. 16A through FIG. 16C). The results further confirmed a stronger dormancy and chemo-resistance of B-ALL cells in the endosteal niche.

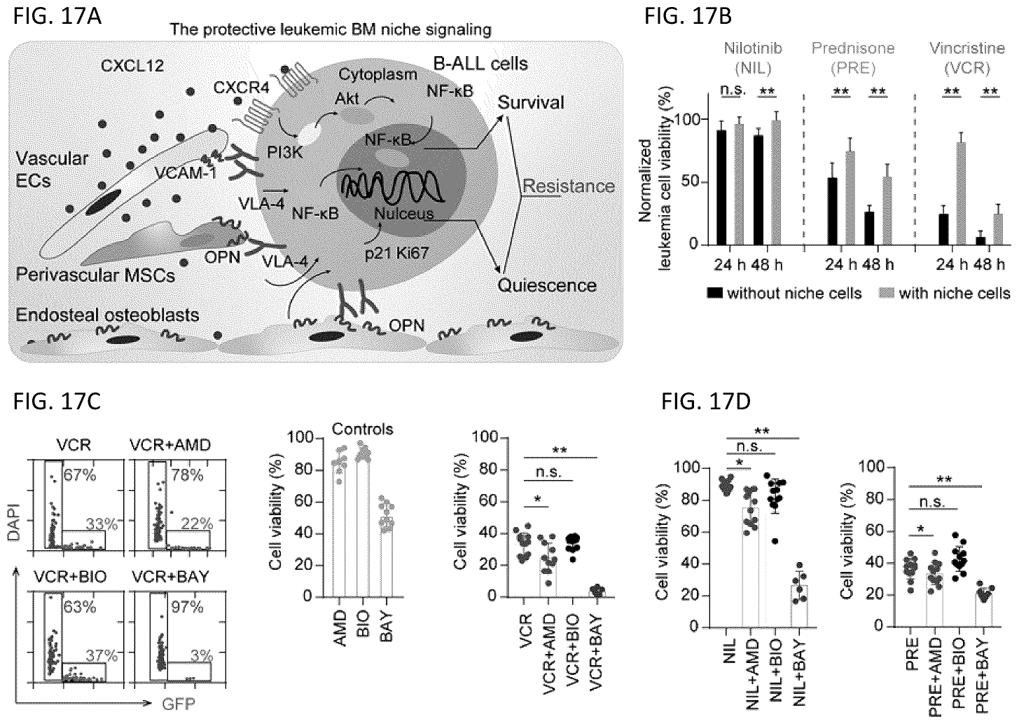

The contributing mechanisms from hematopoietic cells was then investigated. REH B-ALL blasts were cultured with primary human CD34⁺ HSPCs for over one week during which HSPCs may reestablish the hematopoietic environment following the previous protocol (Chou D B et al., Nature biomedical engineering. 2020 April; 4(4): 394-406). The results showed that hematopoietic cells also promoted NF-κB activation in B-ALL cells and chemo-resistance to VCR treatment (FIG. 13I, FIG. 13J). Furthermore, healthy bone marrow mononuclear cells were cultured with and without REH blasts in the leukemia systems for day 1 and day 9 and the number of CD34+ cells was quantified. More interesting, leukemia progression may impede the maintenance and/or expansion of hematopoietic progenitor cells (FIG. 13K, FIG. 13L). Taken together, these findings highlight that BM niche cells, such as ECs, MSCs, and osteoblasts may differentially contribute niche signaling to B-ALL blasts (FIG. 17A), for instance ECs may enhance VCAM-1 singling to regulate B-ALL progression while osteoblasts and MSCs may promote B-ALL dormancy via OPN signaling.

### Eradicating Leukemic Burden by Co-Targeting Leukemia Niche Signaling

To validate the pre-clinical utility of this platform and mechanistic findings (FIG. 17A) in the niche-associated signaling for developing effective therapies, a variety of combinational therapy was screened by co-targeting tumor cell and niche-derived signaling to eradicate nonresponsive leukemia blasts or minimal residual disease burden, a major cause for refractory and relapsed leukemia. Initially, the efficacy of three treatment agents was assessed, i.e. PRE, VCR and NIL. In the absence of niche cells, efficient B-ALL eradication was observed with all three agents in vitro; strikingly, the presence of niche cells resulted in the partial rescue of drug-induced cell death (FIG. 17B), consistent with previous observations (Polak R et al., Blood, The Journal of the American Society of Hematology. 2015 Nov. 19; 126(21): 2404-14; Maffei R et al., haematologica. 2012 June; 97(6): 952).

The following investigation concerned whether co-targeting niche-derived pro-survival signals (CXCR4/CXCL12, VCAM-1/VLA-4, and NF-κB signaling) using either CXCR4 inhibitor (AMD), VLA-4 inhibitor (BIO), or NF-κB inhibitor (BAY) could improve the responsiveness of B-ALL-targeting chemotherapeutics. B-ALL blasts and niche cells (containing ECs and MSCs) were co-cultured for 24 hr in the Leukemia-on-a-Chip, and then administered individual niche-targeting compounds in combination with either NIL, PRE or VCR for 48 hr, after which B-ALL cell viability was measured. Notably, inhibiting CXCR4 and NF-κB signals significantly reversed the chemo-protective activity of niche cells exposed to tumor-targeting agents when compared to vehicle-, AMD- and BAY-treated control groups, whereas VLA-4 inhibition showed no significant effect on the responsiveness of B-ALL blasts to tumor-targeting agents (FIG. 17C, FIG. 17D). The lack of response to VLA-4 treatment may be due to VLA-4 blockade broke the leukemia dormancy to compensate the B-ALL death. This may also indicate a complex and redundant adhesive signaling between leukemia and niche cells, such as CD44 and E-selectin as revealed in HSC niche and other types of leukemia (Winkler I G et al., Nature medicine. 2012 November; 18(11): 1651-7; Jin L et al., Nature medicine. 2006 October; 12(10): 1167-74).

In the present study, a novel 3D microphysiological leukemia BM niche system was engineered for capturing the in vivo pathological features of human and murine B-ALL niche interactions and dissecting the underlying heterogeneous mechanisms regulated by niche cells to drive leukemia progression and chemo-resistance. Unlike solid tumors, a comprehensive understanding of the leukemic BM niche remains in infancy (Duarte D et al., Blood. 2018 Apr. 5; 131(14): 1507-11). The healthy BM niche plays a vital role in regulating HSC fate and maintaining normal hematopoiesis, whereas in hematologic malignancies like acute leukemia, leukemic cells harness the BM niche to favor leukemia survival (Duan C W et al., Cancer cell. 2014 Jun. 16; 25(6): 778-93; Colmone A et al., Science. 2008 Dec. 19; 322(5909): 1861-5). Current in vitro studies adopt suspension cultures of primary leukemia cell lines to test therapeutics, but these simplified methods are clearly inadequate to mirror the complex conditions inside the 3D leukemic BM niche. Pre-clinical murine models allow for an in vivo study of the leukemia-BM niche interactions, however, in vivo complexity may affect reproducibility and accessibility of real-time monitoring of B-ALL interactions with its leukemic niche (Day C P et al., Cell. 2015 Sep. 24; 163(1): 39-53). Microfluidics-based microphysiological systems have been recently reported to reestablish the solid tumor microenvironments, yet limited attempts have been made to precisely replicate the in vivo anatomical structure of the leukemia BM niche and comparatively dissect the heterogeneous leukemia-niche interactions and chemo-resistance mechanisms in B-ALL (Zheng Y et al., Advanced healthcare materials. 2016 May; 5(9): 1014-24; Mannino R G et al., Lab on a Chip. 2017; 17(3): 407-14; Bruce A et al., PLoS One. 2015 Oct. 21; 10 (10): e0140506; Zhang W et al., Tissue Engineering Part C: Methods. 2014 Aug. 1; 20(8): 663-70). The in vitro engineered organotypic Leukemia-on-a-Chip is such a complementary platform to these pre-clinical models as it functions as a bonafide replicate of the in vivo BM tissue architecture. Specifically, it provides several methodological advantages including the capability of control over various biological parameters (e.g. cell type, concentration and composition, tissue architectural information, and extracellular matrix properties), real-time visualization of physiological and pathophysiological dynamics (e.g. cell proliferation and migration, cell fate, and direct and indirect intercellular communications) modulated by internal factors and external stimuli, and the easy setup and compatibility with high throughput on-chip biological assays (e.g. molecular, cellular, and histological characterizations) as well as follow-up cell retrieval for in-depth genetic analyses (e.g. scRNA-seq) (Bhatia S N et al., Nature biotechnology. 2014 August; 32(8): 760-72; Zhang W et al., Tissue Engineering Part C: Methods. 2014 Aug. 1; 20(8): 663-70).

Using this biomimetic niche model, the temporally dynamic interactions were systematically explored between B-ALL blasts and niche cells (i.e. vascular ECs, perivascular MSCs and endosteal osteoblasts) and the distinct roles determined of different niche cells in regulating cytokine (e.g. CXCL12), intercellular adhesive signaling (e.g. VCAM-1 and OPN), and downstream B-ALL pro-survival NF-κB signaling, as well as cell proliferation (i.e. Ki67) and quiescence (i.e. p21) markers, which further demonstrated subtype-associated heterogeneity and treatment responses. The two divergent extrinsic cytokine and intercellular adhesive signaling mechanisms both enhanced downstream leukemia-intrinsic NF-κB signaling, supporting the notion that niche-derived signaling events promote B-ALL survival (Boyerinas B et al., Blood, The Journal of the American Society of Hematology. 2013 Jun. 13; 121(24): 4821-31; Jacamo R et al., Blood. 2014 Apr. 24; 123(17): 2691-702; Hsieh Y T et al., Blood, The Journal of the American Society of Hematology. 2013 Mar. 7; 121(10): 1814-8). Notably, other mechanisms of cytokine signaling may be included in regulating B-ALL progression; for instance, using conventional transwell-based studies, de Rooij et al. found that CCR4/CCL2/CXCL22, CXCR1/2/IL8/GRO-1 and CXCR3/CXCL10 axis were involved in leukemia progression (de Rooij B et al., haematologica. 2017 October; 102(10): e389), which is also confirmed in the present studies. Along with the cytokine signaling, adhesive signaling provided by the niche cells has been reported to promote leukemia progression and therapy resistance. It was confirmed that ECs mainly promote leukemia survival via VCAM-1/VLA-4 axis, while MSCs and osteoblasts may induce leukemia dormancy via OPN signaling. It is notable that the BM microenvironment has a complex cellular composition and orchestrated interactions. The hematopoietic cells, such as monocyte, were also demonstrated to regulate the chemo-resistance of B-ALL and other types of leukemia (Witkowski M T et al., Cancer Cell. 2020 Jun. 8; 37(6): 867-82; Lee Y et al., Blood, The Journal of the American Society of Hematology. 2012 Jan. 5; 119(1): 227-37; Giannoni P et al., haematologica. 2014 June; 99(6): 1078), which requires to be considered in detail to further improve the biomimicry of the system.

The spatial heterogeneity in the leukemic niche was found to increase dormancy in B-ALL cells located at the endosteal niche relative to those in the perivascular niche, suggesting that BM sub-niches may differentially regulate leukemia progression, similar to the observances of its healthy counterpart (Ding L et al., Nature. 2013 March; 495(7440): 231-5; Pinho S et al., Developmental cell. 2018 Mar. 12; 44(5): 634-41). By comparing the engineered BM niches with six types of human B-ALL cells, as well as patient-derived samples, it was revealed that NF-κB signaling is a general niche-derived mechanism to promote leukemia survival. With integration of scRNA-seq, a comprehensive map was generated of the engineered human BM niche for different types of B-ALL blasts (e.g. favorable versus unfavorable), which underpinned that leukemia survival and quiescence across heterogeneous B-ALL subtypes is an orchestration between the microenvironmental cues and tumor genetics. Obtaining such a detailed profile via the biomimetic human leukemic BM stromal niche is of great importance, since to-date it remains a challenge to map the in vivo counterpart, due to the technical difficulties associated with isolating human BM stromal subpopulations in primary leukemic patient BM samples. The current Leukemia-on-a-Chip system, though is not fully a replication of its in vivo counterpart, did provide a useful and powerful way to probe the evolving interactions between BM microenvironment and leukemia, which is presently not available with conventional methods and other blood cancer chips (Zheng Y et al., Advanced healthcare materials. 2016 May; 5(9): 1014-24; Mannino R G et al., Lab on a Chip. 2017; 17(3): 407-14; Bruce A et al., PLoS One. 2015 Oct. 21; 10 (10): e0140506; Zhang W et al., Tissue Engineering Part C: Methods. 2014 Aug. 1; 20(8): 663-70).

Recently, CAR T-cells has emerged as a promising FDA-approved immunotherapy for relapsed and refractory B-ALL (Park J H et al., New England Journal of Medicine. 2018 Feb. 1; 378(5): 449-59), however, patient responses are largely unpredictable. A detailed understanding of the leukemic BM immune niche is also indispensable for improving CAR T-cell therapy. This model is amenable to increases in its biological complexity with addition of patient-derived cells, such as immune cells, to answer how the BM immune niche-derived regulatory signals influence leukemia progression and clinically relevant immune resistance, as well as other key BM niche components (e.g. hematopoietic cells) to interrogate how leukemia pathogenesis hampers normal hematopoiesis and how treatments may restore and maintain homeostasis. Additional work can be directed to recapitulate the biochemical (e.g. oxygen and cytokine gradients) and biophysical (e.g. ECM stiffness and sustained perfusion) cues in the in vivo leukemic BM niche, which may also be involved in regulating leukemia progression and therapy resistance (Duarte D et al., Cell stem cell. 2018 Jan. 4; 22(1): 64-77; Choi J S et al., Science advances. 2017 Jan. 1; 3 (1): e1600455).

### Example 2: Engineering a Personalized Immunotherapy for Leukemia

Chimeric antigen receptor (CAR) T-cell immunotherapy that uses and enhances patients' own T-cells to fight cancer has emerged as an innovative method for treating relapsed and refractory B cell leukemia. Despite the initial promising results, patient responses to this new therapy are variable and 30˜60% of clinical cases unfortunately succumbed to leukemia relapse which largely limits its clinical benefit. While current research has been mostly focused on refining CAR T-cell design, there is a lack of reliable clinical methods to rapidly and accurately assess the potency of these patient-derived CAR T-cell products before administration. Moreover, clinical studies highlight the host immunity as a key role in therapy failure, especially the leukemia immunity in the bone marrow where leukemia initiates and relapse mainly arise. To interrogate CAR T-cell efficiency and relapse mechanisms, the present study engineers a novel “CAR T-on-a-Chip” model as an integrated precision medicine system for a multiparametric and real-time analysis of CAR T-cell functionality in patient-associated bone marrow microenvironments. Overall, this study demonstrates a new paradigm for “clinical trials on a chip” that leads to the development of personalized CAR T-cell immunotherapy strategies with maximized therapeutic efficiency yet minimized relapse potential for leukemia patients.

Therefore, the primary objective of the present study is to develop a novel patient-specific in vitro leukemia CAR T-cell therapy model, termed “CAR T-on-a-Chip”, as an integrated precision medicine system to dissect out the leukemia niche-contributed relapse mechanisms and screen potential responders for a personalized CAR T-cell therapy. To this end, a microfluidics-based leukemia microphysiological system is bioengineered with patient-derived tumor, immune, and niche cells which replicate the in vivo natural pathophysiology of the leukemic bone marrow microenvironment and immunity for a rapid and accurate assessment of CAR T-cell therapy in vitro. The engineered immuno-oncology model is also integrated with label-free nanoplasmonic sensors on-chip for in situ multiplexed monitoring of the dynamic immunological secretomic signatures of CAR T-cell and leukemia immunity that signify CAR T-cell functionality and treatment outcome of either remission, resistance, or relapse.

A CAR T-on-a-Chip model is developed using patient-derived cells to evaluate CAR T-cell functionality (e.g. T-cell extravasation, migration, activation, expansion, and cytotoxicity) and leukemia-immune interactions in the bone marrow over weeks under remission, resistant, relapse conditions. A protocol is standardized to predict patient responses in a patient-specific CAR T-cell therapy model with autologous leukemia patient cells and CAR T-cells, so as to guide the clinical CAR T-cell treatment for relapsed/refractory leukemia patients.

The CAR-T Chip is then applied to interrogate how the host immunity (e.g. immune cell populations, their immunophenotypes, and immune cytokines) and the initial CD19 expression level in different genetic subtypes of B-ALL facilitates disease relapse during CD19 CAR T-cell therapy, as well as to comparatively identify patient-specific or genetic subtype-associated factors involved in the relapse process. With the understanding of the leukemia niche-mediated mechanisms of relapse, CAR T-cell therapy can be preclinically screened and optimized to minimize leukemia relapse by co-targeting key leukemia niche-derived immune-regulatory factors, specifically those gene and signals associated with regulatory T-cell and myeloid cells, to reinvigorate CAR T-cell. Together, the biomimetic in vitro leukemia chip system permits a screening of personalized CAR T-cell immunotherapy with maximized therapeutic efficiency yet minimized relapse potential.

### An In Vitro Leukemia-On-Chip Model for Dissecting the Mechanisms of Chemo-Resistance

To dissect out the chemo-resistance mechanisms of B-ALL, an in vitro organotypic Leukemia-on-a-Chip was engineered to mimic the leukemic BM stromal niche of a high-risk Ph+ B-ALL C57BL/6 mouse model (FIG. 4A) (Ma C et al., Science advances. 2020 Oct. 1; 6 (44): eaba5536). This microfluidics-based microphysiological system replicates the in vivo BM tissue architecture of a central venous sinus, a medullary cavity, and an endosteal region in a 3D fibrin hydrogel. Following a multi-step loading protocol, ECs were seeded in the central sinus region, GFP+ primary murine Ph+ B-ALL cells, ECs and MSCs (OP9, ATCC) stromal niche cells were loaded in the medullary cavity region. The reconstituted leukemic BM niche comprises a vascular network with 3D tube-like structures wrapped around leukemia cells (FIG. 4B, FIG. 4C). Moreover, a mixture of murine osteoblasts (MC3T3, ATCC) and B-ALL cells were also loaded into the outer ring area to form the endosteal region and perivascular/endosteal niche interface (FIG. 4B).

The results demonstrate Leukemia-on-a-Chip resembles the in vivo spatial and cellular composition of the murine BM tissue architecture, and permits a longitudinal monitoring of B-ALL and leukemic BM niche (ECs, MSCs, and osteoblasts) interactions with live-cell imaging. By application of the in vitro engineered biomimetic leukemia BM model, it was determined that CXCL12 cytokine and VCAM-1/OPN adhesive signals from the perivascular and endosteal niches differentially regulate B-ALL dormancy though the NF-kb signaling that confer B-ALL chemo-resistance. The heterogeneity of chemo-resistance across various B-ALL subtypes was further studied by mapping the B-ALL niche signals with scRNA-Seq (FIG. 5B, FIG. 5E).

### Evaluating Human CD19 CAR T-Cell Functionality in Leukemia-On-a-Chip

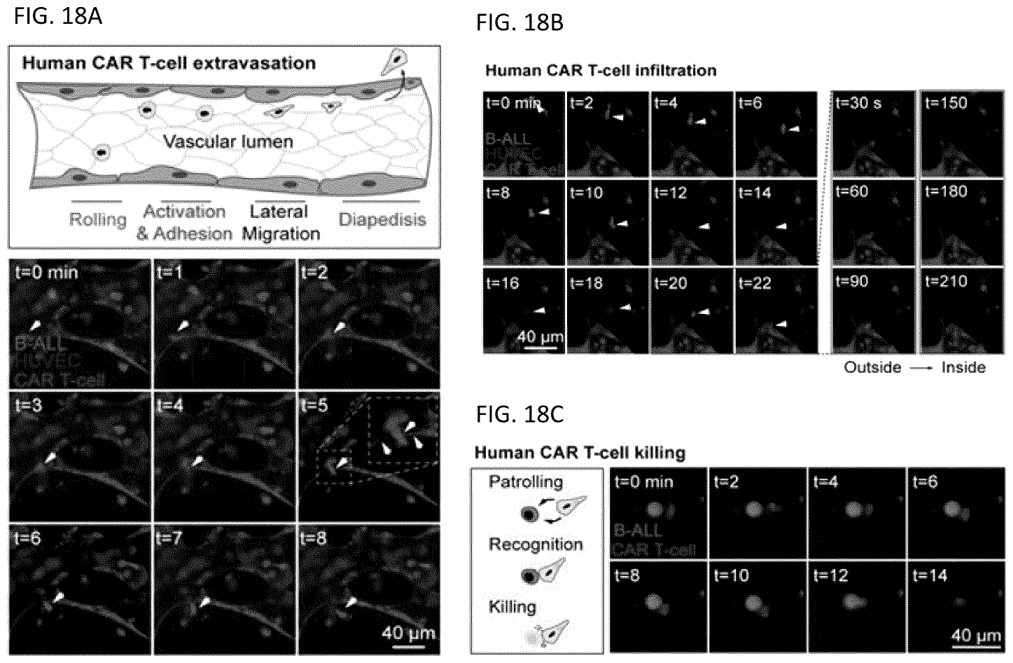

To evaluate human CAR T-cell response, the system was upgraded with human primary umbilical ECs, MSCs, human B-ALL cells (REH, ATCC) and the leukemic immune niche was reconstituted with patient-derived bone marrow mononuclear cells (BMMCs) in a human based ‘Leukemia-on-a-Chip’ microphysiological system. To preclinically analyze the functional capability of CD19 CAR T-cells to eradicate B-ALL cells, CAR T-cells were infused into the vessel of the in vitro leukemic BM niche model containing CD19⁺ or CD19⁻ B-ALL cells. Real-time CAR T-cell extravasation was monitored from the vascular vessel, infiltration in the BM niche, recognition and killing of CD19⁺ B-ALL cells (FIG. 18A through FIG. 18C).

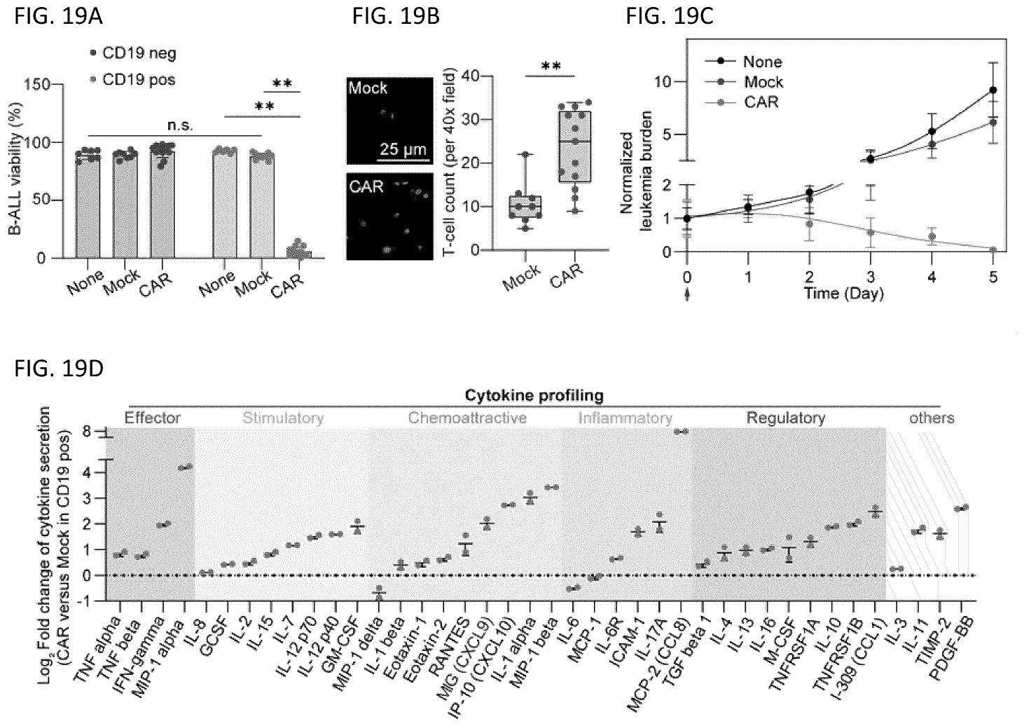

CAR T-cells were found to specifically kill CD19+ B-ALL cells but not CD19⁻ B-ALL cells (FIG. 19A). Moreover, CAR T-cell expanded significantly when being activated by CD19+ leukemia cells (FIG. 19B), and continuously reduce the tumor burden in the leukemia BM niche model (FIG. 19C). Finally, the cytokine milieu was profiled by a membrane-based ELISA kit and a myriad of cytokines was found, functioning in effector, stimulatory, chemoattractive, and inflammatory roles, were released in the leukemia model treated with CAR T-cell as compared to that with Mock T-cell (FIG. 19D). Collectively, a robust in vitro system was established allowing for real-time monitoring of CAR T-cell functionality, which can be applied to study how the leukemic BM immune niche contributes to B-ALL therapy resistance to CAR T-cell, and ultimately disease relapse.

### Label-Free Nanoplasmonic Sensing for Multiplexed Cytokine Detection

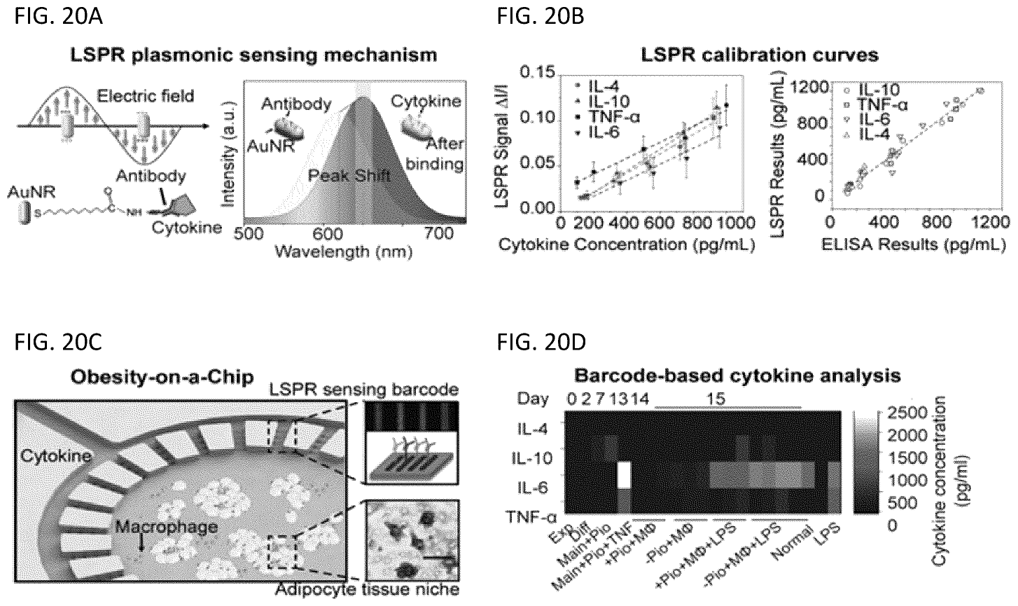

A microfluidic biosensing platform was developed for characterizing different immune subsets for patient “immunophenotyping” (i.e. IL-2, TNF-α from CD8+ T-cells and Macrophages) (Chen W et al., Advanced healthcare materials. 2013 July; 2(7): 965-75; Cui X et al., Lab on a Chip. 2018; 18(3): 522-31). A localized surface plasmon resonance (LSPR)-based nanoplasmonic biosensor enabled rapid, multiplexed, label-free, and in situ cytokine analysis in a biomimetic tissue microenvironment (FIG. 20A through FIG. 20D). The biosensor includes an array of LSPR-based sensing bars on a glass substrate, where a plasmonic phenomenon arises when gold nanorods (AuNRs) are illuminated with light (Chen P et al., Advanced drug delivery reviews. 2015 Dec. 1; 95:90-103; Oh B R et al., ACS sensors. 2016 Jul. 22; 1(7): 941-8; Chen P et al., ACS nano. 2015 Apr. 28; 9(4): 4173-81; Oh B R et al., ACS nano. 2014 Mar. 25; 8(3): 2667-76), for cytokine detection. Multiplexed cytokine absorption and binding events cause specific resonance changes, resulting in distinct intensity and phase shifts of scattering and absorption spectrums (FIG. 20A). The LSPR biosensing unit is capable of measuring target cytokines with concentrations ranging from 100-10,000 pg/mL in a 1 μL sample in a 30-min period, and correlates to gold-standard ELISA assay (FIG. 20B). In a preliminary study, an integrated “inflammatory adipose tissue on-chip” system (FIG. 20C) was demonstrated for the first time to monitor adipocyte differentiation and, equipped with a LSPR biosensor array, to map in situ cytokine profiles on-chip for 15 days with a barcode-based analysis of multiplexed pro-inflammatory (IL-6, TNF-α) and anti-inflammatory (IL-10, IL-4) cytokines in different therapies (Zhu J et al., Lab on a Chip. 2018; 18(23): 3550-60). The ease of fabrication and microfluidic integration and the simple optical scheme of dark-field plasmonic imaging render it an adaptable tool for organ on-chip applications, specifically integrated leukemic BM niche immunophenotyping to evaluate the CAR T-cell-immune-tumor crosstalk.

### Reconstituting the Leukemic BM Niche in an In Vitro Human Leukemia Microphysiological System

To preclinically evaluate human CAR T-cell response, a microfluidics-based in vitro human leukemia microphysiological system was established to mimic the in vivo leukemia BM immunity (FIG. 21A through FIG. 21C). The engineered chip model contains a biomimetic BM central vascular endothelial sinus (center region; FIG. 21A red) and perivascular region with vascular and mesenchymal stromal cells (inner ring; FIG. 21A green) that serve as the interface of leukemia-stroma-immune interactions and integrate the critical features of the in vivo vascularized BM niche. To create a physiologically relevant BM immunity model, human bone marrow mononuclear cells (BMMCs) containing various in vivo BM niche immune cells (e.g. Treg, TAM, and MDSC) from healthy donors (STEMCELL Technologies) and/or clinical patients (AMSBIO) are infused into the device.

### Evaluating CAR T-Cell Functionality in the Engineered Leukemic Immune Niche System

After establishing the B-ALL BM niche, CAR T-cells are infused with different densities and ratios (e.g. 2-5×10⁶ cells/ml; CAR-T:B-ALL=1:1) into the microvessel network inside the leukemic BM niche model. To clearly distinguish different types of cells, CD19-28-1BBζ CAR T-cell (ProMab) and REH B-ALL cells, are pre-labeled with CellTracker Red and DiD dyes (Thermo Fisher). GFP/RFP-expressing HUVECs (Angio-Proteomie) are used to form the microvessel network inside the leukemic BM immune niche model. To evaluate CAR T-cell functional dynamic behaviors within the B-ALL niche, the extravasation and migratory behaviors of CAR T-cell through the BM microenvironment are longitudinally monitored for a period of 2-4 weeks in the device with live cell imaging and confocal microscopy. The T-cell extravasation rate, migration distance and time required for CAR T-cell to reach B-ALL cell are quantified. Moreover, the expression of T-cell activation markers, CD154, CD69, granzyme B (GZMB) and perforin (PFN) are characterized. After interaction with leukemia blasts for 2 days, CAR T-cell activation is demonstrated to be significantly enhanced, where non-engineered (Mock) T-cell remain inactivated.

The cytotoxicity function of the CAR T-cell is characterized in the leukemic BM microenvironment. The tumor apoptosis rate (NucView® 405 Caspase-3 Substrate, Biotium) is longitudinally monitored over 3 weeks in the device to confirm if the tested B-ALL patient can achieve full remission or shows disease relapse after the treatment. The effect of CAR T-cell seeding density is tested on the T-cell activation, expansion rate, the clearance time of tumor cells. In addition, single cell analysis is performed to quantify the killing time and killing cell count for individual CAR T-cells to reveal whether CAR T-cell are functionally homogeneous or heterogeneous and describe the killing pattern of CAR T-cells.

### Personalize CAR T-Cell Therapy Using Patient-Specific In Vitro Leukemia System

A standardized protocol is developed to evaluate and optimize the efficiency of CAR T-cell therapy for individual patients via engineering of patient-specific model using autologous CAR T-cell and patient BM tissues. Patient leukemia and BMMCs (AMSBIO) are infused into the device. Autologous CAR T-cell (ProMab) are produced from the matched patient's PBMCs and these autologous CAR T-cell are infused into the system. To further understand the similarity of in vitro patient-specific system to its in vivo counterpart, scRNA-seq tool is applied to dissect the leukemia immune populations between in vitro and in vivo samples. The established patient-specific microsystem thus will enable a robust and accurate assessment, prediction and screening of optimized CAR T-cell therapy for specific patients.

To further explore the antitumor potency of new generation CAR T-cell products, three types of CAR T-cells are tested: second-generation CAR T-cells carrying CD28 or 4-1BB signal domains and third-generation CAR T-cell with combined 4-1BB and CD28 signaling domains (i.e. CD19-28ζ CAR, CD19-1BBζ CAR, and CD19-28-1BBζ CAR, ProMab) in the B-ALL niche. Previously studies have determined that the two domains exert different functional properties of CAR T-cells, for example, CD28-based CARs direct an immediate antitumor potency, whereas 4-1BB-based CARs have the capacity for long-term persistence (Hamieh M et al., Nature. 2019 April; 568(7750): 112-6). To confirm, the ratio of B-ALL apoptosis is compared when infusing these three types of CAR T-cell after defined time periods (Day 3-14). CD19-28 (and CD19-28-1BBζ CARs have a rapid killing ability compared to CD19-1BBζ CAR at early detection time points, while CD19-1BBζ and CD19-28-1BBζ CARs have continuous killing performance compared to CD19-28ζ CAR at later time points (Day 5 & 7). Therefore, CD19-28-1BBζ CAR T-cells are used following the study as noted otherwise.

### Niche-Contributed Relapse Mechanisms Underlying CAR T-Cell Therapy

The bioengineered CAR-T Chip is applied to interrogate how the initial CD19 expression level and the leukemic BM immunity facilitates leukemia relapse during CD19 CAR T-cell therapy in different genetic subtypes of B-ALL. To correlate with in vivo relapse and remission standard (Fuster J L. Current approach to relapsed acute lymphoblastic leukemia in children. World Journal of Hematology. 2014 Aug. 6; 3(3): 49-70), the percentage of leukemia blast is defined of the total on-chip cell population that is <5% as a remission, while returning to >25% will be consider as a relapse. Theoretically, patient relapse is a randomized phenomenon with a potential relapse rate resulting from statistical analysis of a patient cohort. To achieve enough devices with relapse while minimize total number of repeats different grouping sizes are utilized (such as 6 groups, each with 5 technical repeats or 10 groups, each with 3 technical repeats) with Mann-Whitney test method and a statistical power of 0.95 (Fay D S et al., WormBook: the online review of C. elegans biology. 2013 July: 1-54; Olsen C H, Infection and immunity. 2003 Dec. 1; 71(12): 6689-92). The in vitro system is benchmarked to achieve statistically significant remission and relapse rate (low <20%, medium 20%˜40%, high >40%) similar to the in vivo by tuning the CAR T-cell, immune, and B-ALL cell composition and culture conditions (Porter D L et al., Science translational medicine. 2015 Sep. 2; 7(303): 303ra139; Grupp S A et al., New England Journal of Medicine. 2013 Apr. 18; 368(16): 1509-18; Majzner R G et al., Nature medicine. 2019 September; 25(9): 1341-55).

### Recapitulating CD19 Antigen-Mediated Therapy Relapse in the Engineered Leukemia Niche Model

Multiple mechanisms have been suggested to regulate therapy resistance and leukemia relapse, among them antigen loss, downregulation, genetic mutation of CD19 are known to result in antigen modulated leukemia population that led to treatment failure as CD19⁻ leukemia relapse (Lee D W et al., Blood. 2014 Jul. 10; 124(2): 188-95; Jazirehi A R et al., Immunotherapy: Open Access. 2017; 03(02): 1000142; Feucht J et al., Oncotarget. 2016 Nov. 22; 7(47): 76902). The initiative CD19 expression on leukemia blast may regulate CAR T-cell response, thus differently drive leukemia remission and relapse. To study this in detail, the process of CD19⁻ B-ALL relapse is modeled and validated after CAR T-cell therapy within the CAR-T chip system by using the recently created CD19 knock-out B-ALL cell lines with CRISPR-Cas9 gene editing. Following a chronological observation of CAR T-cell expansion and persistence behaviors and leukemia burden under CD19 CAR T-cell therapy over 3-4 weeks, it is expected that with addition of CD19⁻ leukemia at respective percentages (e.g. 0.1%-1%) the leukemia BM niche system initially demonstrate partial remission then relapse accompanying with expansion of CD19⁻ B-ALL. Further comparative analysis will demonstrate that the higher percentage of CD19⁻ B-ALL cells, the more and faster emergence of leukemia relapse. Also, CD19 expression is screened across six B-ALL lines (REH, SUP-B15, NALM6, RS(4; 11), 697, and UCOB1) to determine if lower of expression of CD19 reduces CD19 CAR T-cell engraftment and exhibits CAR T-Cell resistance and leukemia relapse (Mejstriková E et al., Blood cancer journal. 2017 Dec. 20; 7(12): 1-5).

### Determining the Immune Niche-Contributed Relapse Mechanisms Underlying CAR T-Cell Therapy

Recent evidence suggests that the leukemia BM immune niche potentially contributes to leukemia escape from CAR T-cell therapy (Andersen M H, Leukemia. 2014 September; 28(9): 1784-92). To understand the immune niche-contributed relapse mechanisms, different patient-derived BM immune landscapes are characterized and the host BM immunity (e.g. immune cell populations, their immunophenotypes, and cytokine profiles) in regulating CAR T-cell therapy is dissected with scRNA-Seq mapping and on-chip cytokine profiling, starting with immune cell compositions of B-ALL BM microenvironment using the 10× Genomics scRNA-Seq platform and high-parameter flow cytometry for the in vitro model with or without relapse (Witkowski M T et al., Cancer Cell. 2020 Jun. 8; 37(6): 867-82). All the cells from respective chip models are collected before and after CAR T-cell treatment, as well as BM aspirates from different patient cohorts, and scRNA-Seq analysis is performed on the leukemia blast and immune cell subpopulations. Following the generation of extensive scRNA-Seq data and high-parameter flow cytometry of primary B-ALL BM specimens, extensive bioinformatics analyses is performed in order to identify the niche drivers that may associate with leukemia relapse. These studies demonstrate key leukemia-specific BM niche immune cell populations (e.g. Treg, TAM, and MDSC) and factors that accompany with leukemia progression, remission and relapse. Accumulated suppressor immune cells, e.g. CD16+ non-classical monocytes and Treg, enhance the immunosuppressive cytokine milieu through their dynamic interactions with leukemia blasts in the leukemic BM niche and therefore render CAR T-cell with reduced anti-leukemia potency, which may result in a relapse. It is examined whether depletion of Tregs from the system (Cyclophosphamide, Sigma) (Ghiringhelli F et al., Cancer immunology, immunotherapy. 2007 May 1; 56(5): 641-8) would improve CAR T-cell functions (extravasation, activation, expansion, and persistence) and reduce leukemia relapse. To study the effort of tumor genetics on the BM immunity and CAR T-cell function, scRNA-Seq tool is used to comparatively analyze the cellular population and cytokine pathways between a favorable ETV6-RUNX1 and a high-risk BCR-ABL B-ALL models as well as those from patients of related genetic subtypes.

To complement the scRNA-seq analysis of cellular atlas, integrated, label-free nanoplasmonic sensors on-chip are deployed for in situ multiplexed monitoring of the dynamic immunological secretomic signatures of CAR T-cell and leukemia immunity that signify treatment outcome of either remission, resistance, or relapse. Dynamic cytokine crosstalk between CAR T-cell, B-ALL and niche immune cells are measured in real time by a nanoplasmonic biosensor unit within a surrounding channel (outer ring; FIG. 21A yellow). The biosensor unit includes an array of LSPR-based sensing bars on a glass substrate pre-coated with different cytokine detection antibodies (FIG. 21), where a plasmonic phenomenon arises when gold nanorods are illuminated with light (Chen P et al., Advanced drug delivery reviews. 2015 Dec. 1; 95:90-103; Oh B R et al., ACS sensors. 2016 Jul. 22; 1(7): 941-8; Chen P et al., ACS nano. 2015 Apr. 28; 9(4): 4173-81; Oh B R et al., ACS nano. 2014 Mar. 25; 8(3): 2667-76). A dark-field imaging technique is used to selectively detect intensity changes in LSPR images upon cytokine binding on the gold nanorods for determining cytokine concentrations. The integrated nanoplasmonic sensing bar array permits an in situ, label-free and multiplexed (up to 8 cytokines) cytokine detection in a biomimetic BM microenvironment.

Thus, this integrated sensing system allows for an in situ spatiotemporal mapping of key cytokine mechanisms [i.e. anti-inflammatory (TGF-0, IL-10, M-CSF and CCL2) and pro-inflammatory cytokines in the leukemia niche during CAR T-cell treatment. Cytokine production (IFN-γ, TNF-α, IL-2, and GZMB) is profiled at different time points of CAR T-cell treatment by on-chip LSPR sensing module. The cytokine secretion profiling is compared with scRNA-Seq data to confirm the main cell sources of specific cytokine. INF-γ, TNF-α and IL-2 are significantly increased in CAR T-cell infused groups but not Mock T-cell or no T-cell infused groups. Anti-inflammatory cytokines (e.g. TGF-β, IL-10, M-CSF and CCL2) may induce CAR T-cell exhaustion (enhanced expression of PD-1, TIGIT and LAG3) and dysfunction (reduced expression of activation marker and effector cytokines) and lead to CD19⁺ leukemia relapse with uncontrolled leukemia progression. Different anti-inflammatory cytokines may be targeted by using neutralizing antibodies, 1D11 (anti-TGF-0, R&D), JES052A5 (anti-IL-10, R&D), MAB416 (anti-M-CSF, R&D), and AB-479-NA (anti-CCL2, R&D) alone or in combination to improve CAR T-cell response, such as CAR T-cell infiltration, expansion, and B-ALL apoptosis.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A bone marrow on a chip device, comprising:
a cartridge housing;
a central chamber embedded in the cartridge housing;
at least one aperture fluidly connected to the central chamber; and
a plurality of evenly spaced micropillars arranged in a substantially circular shape within the central chamber such that the central chamber is partitioned into at least a first inner region and a first outer region;
wherein the first inner region comprises endothelial cells configured to mimic a venous sinus and the first outer region comprises endothelial cells and mesenchymal stromal cells configured to mimic a medullary cavity.

2. The device of claim 1, wherein the first inner region and the first outer region are concentric.

3. The device of claim 1, wherein the central chamber comprises an additional second outer region adjacent and concentric to the first outer region, the second outer region being defined by a plurality of evenly spaced micropillars arranged in a substantially circular shape and comprising osteoblasts configured to mimic an endosteal region.

4. The device of claim 1, wherein the plurality of micropillars have a cross-sectional shape selected from the group consisting of: circular, ovoid, square, rectangular, triangular, trapezoidal, and polygonal.

5. The device of claim 1, wherein the plurality of micropillars are evenly spaced by a distance between about 50 μm and 200 μm.

6. The device of claim 1, further comprising one or more sensors comprising capture molecules or probes positioned within the central chamber.

7. The device of claim 6, wherein the capture molecule or probe is selected from the group consisting of: antibodies, antibody fragments, antigens, proteins, nucleic acids, oligonucleotides, peptides, lipids, lectins, inhibitors, activators, ligands, hormones, cytokines, sugars, amino acids, fatty acids, phenols, and alkaloids.

8. The device of claim 6, wherein the one or more sensors are positioned between each of the micropillars.

9. The device of claim 6, wherein the one or more sensors are localized surface plasmon resonance nanoplasmonic biosensors.

10. The device of claim 1, wherein the device is configured to replicate or mimic a bone marrow disease or disorder state selected from the group consisting of: leukemia, myeloma, anemia, infection, poisoning, and physical injury.

11. The device of claim 10, wherein a device replicating or mimicking a leukemia disease state comprises B-cell acute lymphoblastic leukemia (B-ALL) cells in the first outer region.

12. A method of determining leukemia treatment responsiveness, comprising the steps of:
providing the device of claim 11;
administering a leukemia treatment to the central chamber; and
determining leukemia treatment responsiveness based on a measured change in the central chamber.

13. The method of claim 12, wherein the leukemia treatment is a chemotherapeutic selected from the group consisting of: nilotinib, prednisone, vincristine, daunorubicin, doxorubicin, cytarabine, L-asparaginase, 6-mercaptopurine, methotrexate, cyclophosphamide, dexamethasone, and nelarabine.

14. The method of claim 13, wherein the measured change is a quantity of live and dead B-ALL cells after 1-3 days treatment or more.

15. The method of claim 12, wherein the leukemia treatment is chimeric antigen receptor (CAR) T-cell therapy, and wherein the central chamber further comprises CAR T-cells in the first inner region.

16. The method of claim 15, wherein each of the cells in the central chamber are autologous cells.

17. The method of claim 15, wherein the measured change is a percent of leukemia cells relative to total cell population in the central chamber that is 5% or less, indicating responsiveness to CAR T-cell therapy.

18. The method of claim 15, wherein the measured change is a percent of leukemia cells relative to total cell population in the central chamber that is 25% or more, indicating non-responsiveness to CAR T-cell therapy.

19. The method of claim 15, wherein the measured change is a decrease in CD19 expression in B-ALL cells, indicating non-responsiveness to CAR T-cell therapy.

20. The method of claim 15, wherein the measured change is an increase in suppressor immune cells, indicating non-responsiveness to CAR T-cell therapy.

21. The method of claim 15, wherein the measured change is an increase in cytokine levels selected from the group consisting of: IFN-γ, TNF-α, IL-2, and GZMB; indicating responsiveness to CAR T-cell therapy.

22. The method of claim 15, wherein the measured change is an increase in cytokine levels selected from the group consisting of: TGF-β, IL-10, M-CSF, and CCL2; indicating non-responsiveness to CAR T-cell therapy.

23. The method of claim 15, wherein the measured change is an increase in surface markers selected from the group consisting of: CD154, CD69, and CD107a; indicating responsiveness to CAR T-cell therapy.
